SEQUENCE LISTING (25 sequences)

SEQ ID NO: 1 - protein, 604 aa, Homo sapiens
MMRAVWEALAALAAVACLVGAVRGGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGGRCKCNGHAARCVRDRTDSLVCDCRHNTAGPECDRCKPFHYDRPWQRATAREANECVACNCNLHARRCRFNMELYKLSGRKSGGVCLNCRHNTAGRHCHYCKEGYYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKINMKKYCKKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 2 - protein, 604 aa, Mus musculus
MMRAVWEALAALAAVACLVGAVRGGPGLSMFAGQAAQPDPCSDENGHPRRCIPDFVNAAFGKDVRVSSTCGRPPARYCVVSERGEERLRSCHLCNSSDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYYYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGFYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKMNMKKYCRKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 3 - protein, 604 aa, Rattus norvegicus
  MISC_FEATURE (544)..(544): unsure
MMRAVWEALAALAAVACLVGAVRGGPGLSMFAGQAAQPDPCSDENGHPRRCIPDFVNAAFGKDVRVSSTCGRPPARYCVVSERGEERLRSCHLCNSSDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYYYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGFYRDMGKPITHRKACKACDCHPVGAAGKTCNQTTGQCPCKDGVTGITCNRCAKGYQQSRSPIAPCIKIPVAPPTTAASSMEEPEDCDSYCKASKGKLKMNMKKYCRKDYAVQIHILKADKAGDWWKFTVNIISVYKQGTSRIRRGDQSLWIRSRDIAXKCPKIKPLKKYLLLGNAEDSPDQSGIVADKSXLVIQWRDTWARRXRKFQQREKKGKCKKA

SEQ ID NO: 4 - protein, 606 aa, Gallus gallus
MPRRGAEGPLALLLAAAWLAQPLRGGYPGLNMFAVQTAQPDPCYDEHGLPRRCIPDFVNSAFGKEVKVSSTCGKPPSRYCVVTEKGEEQVRSCHLCNASDPKRAHPPSFLTDLNNPHNLTCWQSDSYVQYPHNVTLTLSLGKKFEVTYVSLQFCSPRPESMAIYKSMDYGKTWVPFQFYSTQCRKMYNKPSRAAITKQNEQEAICTDSHTDVRPLSGGLIAFSTLDGRPTAHDFDNSPVLQDWVTATDIKVTFSRLHTFGDENEDDSELARDSYFYAVSDLQVGGRCKCNGHASRCVRDRDDNLVCDCKHNTAGPECDRCKPFHYDRPWQRATAREANECVACNCNLHARRCRFNMELYKLSGRKSGGVCLNCRHNTAGRHCHYCKEGFYRDLSKPISHRKACKECDCHPVGAAGQTCNQTTGQCPCKDGVTGITCNRCAKGYQQSRSPIAPCIKIPAAPPPTAASSTEEPADCDSYCKASKGKLKINMKKYCKKDYAVQIHILKAEKNADWWKFTVNIISVYKQGSNRLRRGDQTLWVHAKDIACKCPKVKPMKKYLLLGSTEDSPDQSGIIADKSSLVIQWRDTWARRLRKFQQREKKGKCRKA

SEQ ID NO: 5 - protein, 934 aa, Homo sapiens
MGARSGARGALLLALLLCWDPRLSQAGTDSGSEVLPDSFPSAPAEPLPYFLQEPQDAYIVKNKPVELRCRAFPATQIYFKCNGEWVSQNDHVTQEGLDEATGLRVREVQIEVSRQQVEELFGLEDYWCQCVAWSSAGTTKSRRAYVRIAYLRKNFDQEPLGKEVPLDHEVLLQCRPPEGVPVAEVEWLKNEDVIDPTQDTNFLLTIDHNLIIRQARLSDTANYTCVAKNIVAKRRSTTATVIVYVNGGWSSWAEWSPCSNRCGRGWQKRTRTCTNPAPLNGGAFCEGQAFQKTACTTICPVDGAWTEWSKWSACSTECAHWRSRECMAPPPQNGGRDCSGTLLDSKNCTDGLCMQMLEASGDAALYAGLVVAIFVVVAILMAVGVVVYRRNCRDFDTDITDSSAALTGGFHPVNFKTARPSNPQLLHPSVPPDLTASAGIYRGPVYALQDSTDKIPMTNSPLLDPLPSLKVKVYSSSTTGSGPGLADGADLLGVLPPGTYPSDFARDTHFLHLRSASLGSQQLLGLPRDPGSSVSGTFGCLGGRLSIPGTGVSLLVPNGAIPQGKFYEMYLLINKAESTLPLSEGTQTVLSPSVTCGPTGLLLCRPVILTMPHCAEVSARDWIFQLKTQAHQGHWEEVVTLDEETLNTPCYCQLEPRACHILLDQLGTYVFTGESYSRSAVKRLQLAVFAPALCTSLEYSLRVYCLEDTPVALKEVLELERTLGGYLVEEPKPLMFKDSYHNLRLSLHDLPHAHWRSKLLAKYQEIPFYHIWSGSQKALHCTFTLERHSLASTELTCKICVRQVEGEGQIFQLHTTLAETPAGSLDTLCSAPGSTVTTQLGPYAFKIPLSIRQKICNSLDAPNSRGNDWRMLAQKLSMDRYLNYFATKASPTGVILDLWEALQQDDGDLNSLASALEEMGKSEMLVAVATDGDC

SEQ ID NO: 6 - DNA, 25 bp, artificial sequence
  antisense
cgccttcctcagcctctcctgtgct

SEQ ID NO: 7 - DNA, 25 bp, artificial sequence
  antisense
catttaaccggctcgtacctgcatg

SEQ ID NO: 8 - DNA, 19 bp, artificial sequence
  primer
actagaatgctgtccagac

SEQ ID NO: 9 - DNA, 18 bp, artificial sequence
  primer
agaggagagcaacggatg

SEQ ID NO: 10 - DNA, 19 bp, artificial sequence
  primer
tgcacatgctttacgtgtg

SEQ ID NO: 11 - DNA, 17 bp, artificial sequence
  primer
cgcgtgtcgtgttgcac

SEQ ID NO: 12 - DNA, 20 bp, artificial sequence
  primer
agctgtcccttaatgctggt

SEQ ID NO: 13 - DNA, 20 bp, artificial sequence
  primer
aaggctgtgtacataaggcc

SEQ ID NO: 14 - DNA, 20 bp, artificial sequence
  primer
actggatctttcagctcaag

SEQ ID NO: 15 - DNA, 20 bp, artificial sequence
  primer
agtaattcaggtaccggtcc

SEQ ID NO: 16 - DNA, 20 bp, artificial sequence
  primer
atttgccgctgctggatcct

SEQ ID NO: 17 - DNA, 20 bp, artificial sequence
  primer
acaacaaaccgtccacagct

SEQ ID NO: 18 - DNA, 20 bp, artificial sequence
  primer
gcctcgagtacttggtaagt

SEQ ID NO: 19 - DNA, 20 bp, artificial sequence
  primer
tgtgtcattctctgtaggcc

SEQ ID NO: 20 - DNA, 20 bp, artificial sequence
  primer
aacactctcagtggaccgag

SEQ ID NO: 21 - DNA, 20 bp, artificial sequence
  primer
tccttaactgagtggtcctg

SEQ ID NO: 22 - DNA, 20 bp, artificial sequence
  primer
ctatgcttaccggaaccgag

SEQ ID NO: 23 - DNA, 20 bp, artificial sequence
  primer
accatgcccggccgaataat

SEQ ID NO: 24 - DNA, 20 bp, artificial sequence
  primer
gcttcaaggttggcatcaac

SEQ ID NO: 25 - DNA, 20 bp, artificial sequence
  primer
tagtattcctctccttcttc

(19) United States
(12) Patent Application Publication
(10) Pub. No.: US 2006/0153840 A1
(43) Pub. Date: Jul. 13, 2006

(54) Methods for preventing or treating a condition or a disease associated with angiogenesis

(76) Inventors: Anne Eichmann, Paris (FR); Jean-Leon Thomas, Paris (FR); Xiaowei Lu, Stanford, CA (US); Marc Tessier-Lavigne, Woodside, CA (US)

Correspondence Address: JACOBSON HOLMAN PLLC, 400 SEVENTH STREET N.W., SUITE 600, WASHINGTON, DC, 20004, US

(21) Appl. No.: 11/033,476

(22) Filed: Jan. 12, 2005

(51) Int. Cl. A61K 39/395

(52) U.S. Cl. 424/143.1

(57) ABSTRACT

A method for regulating angiogenesis in a subject in need thereof, comprising administering to said subject an effective amount of a substance capable of modulating the activity of a netrin-1 receptor.

### FIELD OF THE INVENTION

The present invention relates generally to compositions and methods for modulating angiogenesis. More particularly, the invention pertains to compositions and methods for modulating angiogenesis mediated by netrin-1 or the netrin-1 receptor. This invention further relates to methods for the screening of substances of therapeutically useful for preventing or treating conditions and diseases associated with angiogenesis.

### BACKGROUND OF THE INVENTION

Angiogenesis is a biological process of generating new blood vessels into a tissue or an organ. Under normal physiological conditions, humans or animals undergo angiogenesis only in very specific restricted situations. For example, angiogenesis is normally observed in wound healing, fetal and embryonic development and formation of the corpus luteum, endometrium and placenta. It is widely accepted that new vessel growth is tightly controlled by many angiogenic regulators and the switch of the angiogenesis phenotype depends on the net balance between up-regulation of angiogenic stimulators and down-regulation of angiogenic suppressors. The control of angiogenesis has been found to be altered in certain disease states and, in many cases, the pathological damage associated with the disease is related to the uncontrolled angiogenesis.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

In disease states where an imbalance of the angiogenic process is encountered, increasing or inhibiting angiogenesis could avert the corresponding body damages.

Persistent, unregulated angiogenesis occurs in a multiplicity of disease states, including tumor growth and tumor metastasis, and supports the pathological damage seen in these conditions. The diverse pathological states that are due to unregulated angiogenesis have been grouped together as angiogenic dependent or angiogenic associated diseases or conditions.

One example of a disease mediated by angiogenesis is ocular neovascular disease, which is characterized by invasion of new blood vessels into the structure of the eye such as the retina or the cornea. It is the most common cause of blindness and is involved in approximately twenty eye diseases. In age-related macular degeneration, the associated visual problems are caused by an ingrowth of chorioidal capillaries through defects in Bruch's membrane with proliferation of fibrovascular tissues beneath the retinal pigment epithelium. Angiogenic damage is also associated with diabetic retinopathy, retinopathy of prematurity, corneal graft rejection, neovascular glaucoma, and retrolental fibroplasias. Other diseases associated with corneal neovascularization include, but are not limited to, epidemic keratocunjunctivitis, Vitamin A deficiency, contact lens overwear, atopic keratitis, superior limbic keratitis, pterygium kreatitis sicca, sjogrens, acne rosacea, phylectenulosis, syphilis, mycobacterial infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, rheumatois arthritis, systemic lupus, polyarteritis, Wegener's sarcoidiosis, scleritis, Stevens-Johnson disease, pemphigoid, radial keratotomy and corneal graft rejection.

Diseases associated with retinal/choroidal neovascularization include, but are not limited to, diabetic retinopathy, macular degeneration, sickle cell anemia, sracoid, syphilis, pseudoxanthoma elasticum, Paget's disease, vein occlusion, artery occlusion, carotid obstructive disease, chronic uveitis/vitritis, mycobaterial infections, Lyme's disease, systemic lupus erythematosis, retinopathy of prematurity, Eale's disease, Bechet's disease, infections causing retinitis or choroiditis, presumed ocular histoplasmosis, Best's disease, myopia, optic pits, Stargardt's disease, pars planitis, chronic etinal detachment, hyperviscosity syndromes, toxoplasmosis, trauma and post-laser complications. Other diseases include, but are not limited to, diseases associated with rubeosis, which causes neovascularisation of the angle, and diseases caused by abnormal proliferation of fibrovascular or fibrous tissue including all forms of proliferative vitreoretinopathy.

Another disease in which angiogenesis is involved is rheumatoid arthritis. The blood vessels in the synovial lining of the joints undergo angiogenesis. In addition to forming new vascular networks, the endothelial cells release factors and reactive oxygen species that lead to panus growth and cartilage destruction. The factors involved in angiogenesis may actively contribute to, and help maintain, the chronically inflamed state of rheumatoid arthritis.

Angiogenesis is also involved in osteoarthritis. The activation of the chondrocytes by angiogenic-related factors contributes to the destruction of the joint.

Pathological angiogenesis is also involved in chronic inflammation. Such disease states as ulcerative colitis and Crohn's disease show histological changes with the ingrowth of new blood vessels into inflamed tissues. Illustratively, Bartonellosis, a bacterial infection found in South America, can result in a chronic stage that is characterized by proliferation of vascular endothelial cells.

Another pathological role associated with angiogenesis is found in atherosclerosis, wherein the plaques formed within the lumen of blood vessels have been shown to possess angiogenic stimulatory activity.

Also, deregulation of angiogenesis is the cause of hemangioma, which is one of the most frequent angiogenic diseases in childhood.

Deregulation of angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels.

Angiogenesis is also involved in normal physiological processes such as reproduction and wound healing. Angiogenesis is an important step in ovulation and also in implantation of the blastula after fertilization. Prevention of angiogenesis could be used to induce amenorrhea, to block ovulation or to prevent implantation by the blastula.

In wound healing, excessive repair or fibroplasias can be a detrimental side effect of surgical procedures and may be caused or exacerbated by angiogenesis. Adhesions are a frequent complication of surgery and lead to problems such as small bowel obstruction.

Of particular importance, both primary and metastatic tumors need to recruit angiogenic vessels for their growth. If this angiogenic activity could be repressed or eliminated, then the tumor would not grow. Thus, angiogenesis is prominent in solid tumor formation and metastasis.

Angiogenic factors have been found associated with various solid tumors such as rhabdomyosarcomas, retinoblastoma, Ewing sarcoma, neuroblastoma, osteosarcoma as well as with colorectal cancer. Tumors in which angiogenesis is important include solid tumors, and benign tumors such as acoustic neurona, neurofibroma, trachoma and pyogenic granulomas. Further, angiogenesis has been associated with blood-born tumors such as leukemias, any of various acute or chronic neoplastic diseases of the bone marrow in which unrestrained proliferation of white blood cells.

Angiogenesis is important in two stages of tumor metastasis. The first stage where angiogenesis is important is in the vascularization of the tumor which allows tumor cells to enter the blood stream and to circulate throughout the body. After the tumor cells have left the primary site, and have settled into the secondary, metastasis site, angiogenesis must occur before the new tumor can grow and expand.

As illustrated hereabove, deregulation of angiogenesis is the cause of a wide variety of pathological conditions or disease states.

In some situations promoting or up-regulating angiogenesis is sought, for example to treat a patient with a disease or a condition that is indicated by decreased vascularization, and also when a rapid wound healing is sought. Other conditions where promotion of angiogenesis is desired include, without being limited to, peripheral vascular disease, hypertension, inflammatory vasculitides, Reynaud's disease and Reynaud's phenomenon, aneurysms, arterial restenosis, thrombophlebitis, lymphangitis, lymphedema, wound healing and tissue repair (especiall hepatic and renal tissues), ischemia reperfusion injury, angina, myocardial infarctions such as acute myocardial infarctions, chronic heart conditions, heart failure such as congestive heart failure, and osteoporosis.

In many other situations, particularly when preventing or treating caners is sougth, a down-regulation of angiogenesis is desired.

Various substances are already known that prevent deregulation of angiogenesis, most of them inhibiting angiogenesis.

Until now, at least ten endogenous angiogenic inhibitors have been identified in the art. One such molecule is angiostatin, which consists of the plasminogen kringle I through IV. Also, apolipoprotein (a), one of the proteins having kringle structures, is a candidate for a novel angiogenesis inhibitor.

Several other kinds of compounds have been used to prevent angiogenesis. For example, Taylor et al. have used protamine to inhibit angiogenesis, although its toxicity limits its practical use as a therapeutic agent (Taylor et al., 1982, Nature, Vol. 297:307). Folkman et al. have disclosed the use of heparin and steroids to control angiogenesis (Folkman et al., 1983, Science, Vol. 221: 719). Other agents which have been used to inhibit angiogenesis include ascorbic acid ethers and related compounds (Japanese Patent Kokai Tokkyo Koho N^{o} 58-131978). Also, a fungal product, fumagillin, is a potent angiostatic agent in vitro, as well as its synthetic derivative O-substituted fumagillin.

Recently, the United States Food and Drug Administration has granted the first marketing authorization for an anti-angiogenic therapeutical agent, which is termed bevacizumab. Bevacizumab is an humanized antibody directed against the angiogenic factor VEGF. Bevacizumab prevents the binding of VEGF to its effector receptor and has been initially used for treating colorectal cancer.

When taking into account the wide diversity of conditions or diseases that are caused by a deregulation of angiogenesis, or where deregulation of angiogenesis is involved, as well as the severity of several of these diseases, it flows that here is a high need in the art for the provision of novel therapeutically active substances that would allow circumventing physiological situations associated with a deregulation of angiogenesis, typically pathologically strong angiogenesis activity, and especially in cancer.

There is also a need in the art for new methods that would enable the screening of candidate substances for their angiogenesis regulation potency.

### SUMMARY OF THE INVENTION

The present invention relates to a method for inhibiting or increasing angiogenesis in a patient in need thereof, said method comprising a step of administering to said patient an effective amount of a substance that modulates positively or negatively the netrin-1 receptor activity.

It also relates to the use of a substance that modulates positively or negatively the netrin-1 receptor activity for manufacturing a pharmaceutical composition for inhibiting or increasing angiogenesis.

The invention also pertains to methods for the screening of candidate substance for its anti-angiogenic activity.

It also concerns methods for the screening of substances that increases angiogenesis.

### BRIEF DESCRIPTION OF THE DRAWINGS

FIG. 1: Unc5h2 is expressed in the vascular system. a-d. In situ hybridization, E12.5 neural tube (a, b) and eye (c, d). Note Dcc expression in the dorsal neural tube (NT) (a), Unc5h1 expression in the retina (r) (c) and Unc5h2 expression in capillaries (arrows, b, d). I: lens. Bars: 0.1 mm. e: X-gal staining, E10.5 Unc5h2±. Note expression in dorsal aorta (a), internal carotid artery (ica) and intersomitic arteries (arrowheads). Neural expression of Unc5h2 is restricted to the otic placode (o) and eye (e). h: heart. Bar: 0.5 mm. f: section of X-gal stained E12.5±embryo. Dorsal aorta (ao) and its branches are labeled; cardinal vein (cv) is negative. v: vertebra. g, h: lacZ (g)-isolectinB4 (h) double labeling of retinal vasculature, P4 Unc5h2±. Arteries (A), but not veins (V) express Unc5h2. i: isolectinB4 (red)-anti-beta-galactosidase antibody (green) double-labeling. Weakly isolectinB4-positive pericytes lining the arterial stem (arrows) are Unc5h2-negative. Arrowhead: Endothelial tip cell is beta-galactosidase positive. j: X-gal positive endothelial tip cell. Bars: f-h: 0.1 mm, i, j: 10 μm.

FIG. 2: Increased capillary branching and tip cell filipodia extension in Unc5h2 mutant embryos.

a-f: Whole-mount X-gal staining of E10.5 Unc5h2± and −/− littermates. Homozygous embryos show increased vessel branching from the internal carotid artery (ica) (b, arrows), in the somitic region (s) (d, arrows) and in the neural tube (e, f). da: dorsal aorta. Bars: 0.2 mm. g, h: lacZ-isolectinB4 double staining of E11 neural tube capillaries. Note double staining in virtually all endothelial cells. Arrows: filopodial extension from tip cells is increased in −/− capillaries (h). i-k: isolectinB4 staining of E12 hindbrain vessels. I: quantification of number of branch points/photomicrograph. m, n: high-magnification of E10.5 tip cells extending at the midline (asterisk). Note increased filopodial extension in −/−capillaries (arrows). Bars: g-k 0.1 mm, m, n 50 μm.

FIG. 3 : Abnormal morphology of Unc5h2 mutant arteries.

a, b. PECAM-1 staining, tail region, E11. Collapsed lumen of aorta (ao) branch in −/−embryo (arrow, b). Cardinal vein (cv) appears normal. c-f. In situ hybridization. In spite of abnormal artery lumen (arrows, d, f), −/−arteries express Nrp-1 (d) and Pdgfr-β (f) normally. g-k: Normal proliferation of Unc5h2 mutant vessels. g, h, j, k: BrdU (yellow)-lacZ (black) double stainings. h, k: higher magnification of boxed regions in g, j. Note abnormal vessel lumen in −/−intersomitic artery j, k, arrow). i: quantification of BrdU-lacZ double-labeled endothelial cells. j. Caspase-3 (green)-isolectin-B4 (red) double staining of WT hindbrain vessels. Absence of endothelial cell apoptosis. Bars: 0.1 mm.

FIG. 4 : Netrin-1 reduces endothelial cell migration and filopodial extension in vitro.

a, b. Expression of Netrin receptors and □-tubulin (bottom band) in HUAEC (a) or HUVEC (b). c. Transwell migration assay. d. Wound migration assay using HUAEC. c, d each shows representative of three experiments performed in duplicate wells. Migration is reduced by Netrin-1. e-h: aortic ring sprouting assay. Still images from time-lapse videos taken at the indicated time points. Endothelial tip cell filopodia are indicated (arrowheads). Note little net movement of untreated tip cells (e, f). g, h: exposure of two tip cells (1, 2) to a Netrin-1 gradient (source indicated by arrows) induces rapid filopodial retraction and backward movement of both cells. Representative of three experiments. Bars: 30 μm.

FIG. 5 : Netrin-1 provokes filopodial retraction of endothelial cells in vivo.

Confocal images of isolectin-B4 stained tip cells (arrowheads) at the angiogenic front from control (a) or eyes injected with the indicated proteins. Note filopodial retraction in b, e, f. Bars: 50 μm.

FIG. 6 : Netrin-1-induced filopodial retraction is lost in the absence of Unc5h2.

Confocal images, E10.5 hindbrain whole-mount isolectinB4 stained capillaries after injection of recombinant proteins as indicated. a-d: dorsal angiogenic front. Arrowheads: tip cells extending filopodia. e: counting of tip cells/photomicrograph, note 30% reduction in Netrin-1-injected wild-type embryos; −/−embryos show no significant reduction in tip cell number. f-j: Higher magnifications of filopodial extension from tip cells. Bars: a-d 100 μm, f, g 50 μm, h-j 20 μm.

### DETAILED DESCRIPTION OF THE INVENTION

The present invention relates to novel methods and compositions for preventing or treating conditions and diseases associated with a deregulation of angiogenesis, that make use of substances that modulate the netrin-1 receptor activity. It also pertains to methods for the screening of substances that modulate the netrin-1 receptor activity that are useful for preventing or treating conditions and diseases associated with a deregulation of angiogenesis in a human or an animal in need thereof.

The present invention is based on the inventors's supprising findings that netrin-1 signaling is involved in the regulation of angiogenesis both in vitro and in vivo. Without being limited to specific theories or mechanisms, the present invention is based on the particular finding that Netrin-1, acting via its receptor UNC5B, can be a negative regulator of capillary branching during the early stage of angiogenesis, when proliferating endothelial cells sprout and branch out to form the highly stereotyped vascular network. It has been found according to the invention that netrin-1 inhibits migration of human umbilical artery endothelial cells (HUAECs) and induces the retraction of tip cell filopodia and backward movement of tip cells. Further, it has been found that netrin-1 induces in vivo a marked decrease in filopodial extension over the entire angiogenic front of the vasculature. Moreover, the netrin-1-induced angiogenesis down-regulation could be reversed by blocking availability of netrin-1 to its receptor, such as UNC5H2; and inhibiting or blocking the netrin-1 receptor activity can induce strong angiogenesis.

Therefore, in one aspect, invention encompasses a method for regulating angiogenesis in a patient in need thereof, said method comprising a step of administering to said patient an effective amount of a substance that modulates positively or negatively the netrin-1 receptor activity.

According to this aspect, the invention pertains to the use of a substance that modulates positively or negatively the netrin-1 receptor activity for manufacturing a pharmaceutical composition for inhibiting or increasing angiogenesis in a patient in need thereof.

As used herein, the term “netrin-1” refers to any netrin-1 protein, including mammal netrin-1 protein, like human netrin-1, as well as human netrin-1 orthologue proteins from other mammals including those originating from Mus, Rattus and Gallus mammal species. Thus, netrin-1 encompasses the Homo sapiens netrin-1 of SEQ ID No.1, the Mus musculus netrin-1 of SEQ ID No. 2, the Rattus norvegicus netrin-1 of SEQ ID No.3 and the Gallus gallus netrin-1 of SEQ ID No.4. Netrin-1 proteins are described by, for example, Leonardo et al. (1997, Cold Spring Harb. Symp. Quant. Biol., Vol. 62 : 467478) and Serafini et al. (1994, Cell, Vol. 78(3): 409424).

Also encompassed are any variant of a netrin-1 protein, including any variant of the netrin-1 proteins selected from the group consisting of SEQ ID Nos. 1, 2, 3 and 4.

As used herein, the term “netrin-1 receptor” refers to any netrin-1 receptor protein, including but not limited to mammalian netrin-1 receptor protein, such as human netrin-1 receptor protein. Preferably, the netrin-1 receptor of the invention is expressed on the surface of vascular endothelial cells; more preferably, the netrin-1 receptor is UNC5B, also known as UNC5H2. Thus, the netrin-1 receptor encompasses the Homo sapiens netrin-1 receptor of SEQ ID No.5.

Any variant of a netrin-1 receptor protein, including any variant of the netrin-1 receptor protein of SEQ ID No.5, is also encompassed by the present invention.

As intended herein, a “variant” from netrin-1 or from the netrin-1 receptor refers to a polypeptide having at least 80% amino acid identity with the reference netrin-1 or the reference netrin-1 receptor. Alternatively, a variant polypeptide possesses at least 81%, 82%, 83%, 84%, 85%, 86%, 87%, 88%, 89%, 90%, 91%, 92%, 93%, 94%, 95%, 96%, 97%, 98% or 99% amino acid identity with the reference netrin-1 or the reference netrin-1 receptor.

“Percent (%) amino acid sequence identity” with respect to the reference polypeptide sequences identified herein is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST and BLAST-2, software. Those skilled in the art can determine appropriate parameters for measuring alignment, including any algorithms needed to achieve maximal alignment over the full-length of the sequences being compared.

Preferably, percentage of amino acid sequence identity is determined using the sequence comparison program NCBI-BLAST2 (Altschul et al., Nucleic Acids Res., 25:3389-3402 (1997)). The NCBI-BLAST2 sequence comparison program may be downloaded from http://www.ncbi.nlm.nih.gov. or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, drop off for final gapped alignment=25 and scoring matrix=BLOSUM62.

According to one embodiment of the present invention, it is contemplated that a substance capable of modulating positively the netrin-1 receptor activity is useful in a method for inhibiting angiogenesis and disorder associated therewith. Thus, the invention relates to a method for inhibiting angiogenesis in a subject in need thereof, comprising administering to said subject an effective amount of a substance capable of promoting the activity of a netrin-1 receptor.

The present invention further relates to the use of a substance capable of promoting the netrin-1 receptor activity in the manufacture of a pharmaceutical composition for inhibiting angiogenesis.

As used herein, a substance capable of modulating positively or promoting the netrin-1 receptor activity refers to a substance that upregulates, increases, enhances or mimics the netrin-1 receptor activity. In one aspect the substance binds to the netrin-1 receptor and induces netrin-1 receptor-expressing cells to a decrease in cell migration or to a decrease in filopodial extension. Such anti-angiogenic activity of said substance can be easily assessed by the one skilled in the art, for instance through the various in vitro and in vivo assays that are disclosed in the examples.

Various substances that bind to the netrin-1 receptor and inhibit angiogenesis are described further in the present specification. Many or all of these substances may be selected by carrying out the screening methods that are described below.

In another aspect, the present invention relates to a method for promoting angiogenesis in a subject in need thereof, comprising administering to said subject an effective amount of a substance capable of negatively modulating the activity of a netrin-1 receptor.

As used herein, a substance that negatively modulates or inhibits the netrin-1 receptor activity refers to a substance that downregulates, blocks, decreases or antagonizes the netrin-1 receptor activity. For example, it can be a substance that prevents the binding of netrin-1 to the netrin-1 receptor or (ii) a substance that affects production of the netrin-1 receptor by the netrin-1 receptor-expressing cells. These substances capable of negatively modulating the netrin-1 receptor activity possess angiogenesis-promoting activity.

Various substances that promote angiogenesis by inhibiting the netrin-1 receptor activity are described further in the present specification. Many or all of these substances may be selected by carrying out the screening methods that are described below.

Screening Methods According to the Invention

This invention encompasses methods for screening candidate substances for their ability to bind to the netrin-1 receptor and to inhibit angiogenesis, thus methods for screening substances that mimic the anti-angiogenic activity of netrin-1 when bound on the netrin-1 receptor.

The assays can be performed in a variety of formats, including protein-protein binding assays, biochemical screening assays, immunoassays, and cell-based assays, which are well characterized in the art.

The most part of the assays for candidate substances are common in that they call for contacting the drug candidate with a netrin-1 receptor polypeptide under conditions and for a time sufficient to allow these two components to interact.

Substances that positively modulate the netrin-1 receptor activity also include those substances that alter the expression of a functional netrin-1, preferably locally at proximity to the vascular endothelial cells. The latter substances encompass netrin-1-specific anti-sense polynucleotides, such as those that will be described further in the present description.

This invention also encompasses methods for the screening of candidate substances for their ability to prevent the binding of netrin-1 to the netrin-1 receptor, the positively selected candidate substances being potentially endowed with angiogenesis-promoting activity. Screening for candidate substances having angiogenesis-promoting activity may be performed with the same screening methods as those carried out for screening anti-angiogenic substances, since, in both cases, detection of the binding of a molecule on the netrin-1 receptor is performed.

For screening substances having angiogenesis-promoting activity, what is measured is the binding between netrin-1 and the netrin-1 receptor, and more precisely the ability of the pro-angiogenic candidate substance to affect the binding between netrin-1 and the netrin-1 receptor.

Thus, screening for anti-angiogenic substances include the use of two partners, through measuring the binding between two partners, respectively the netrin-1 receptor and the candidate compound.

Further, screening for pro-angiogenic substances include the use of three partners, through measuring the alteration of the binding between two partners, respectively netrin-1 and the netrin-1 receptor, by the third partner which comprises the pro-angiogenic candidate substance.

Substances that negatively modulate the netrin-1 receptor activity also include those substances that alter the expression of a functional netrin-1 receptor at the surface of vascular endothelial cells. The latter substances encompass netrin-1 receptor-specific anti-sense polynucleotides, such as those that will be described further in the present description.

A. Binding Assays

Compounds that interfere with the interaction of a gene encoding a netrin-1 receptor polypeptide identified herein can be tested as follows: usually a reaction mixture is prepared containing the netrin-1 receptor polypeptide under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the candidate substance and the netrin-1 receptor polypeptide present in the mixture is monitored as described hereinabove. The formation of a complex in the control reaction(s) but not in the reaction mixture containing the test compound indicates that the test compound interferes with the interaction of the test compound and its reaction partner.

In binding assays, the interaction is binding and the complex formed can be isolated or detected in the reaction mixture. In a particular embodiment, the netrin-1 receptor polypeptide or the drug candidate is immobilized on a solid phase, e.g., on a microtiter plate, by covalent or non-covalent attachments. Non-covalent attachment generally is accomplished by coating the solid surface with a solution of the netrin-1 receptor polypeptide and drying. Alternatively, an immobilized antibody, e.g., a monoclonal antibody, specific for the netrin-1 receptor polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a Is labeled antibody specifically binding the immobilized complex.

If the candidate compound interacts with but does not bind to a particular netrin-1 receptor polypeptide, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340: 245-246 (1989); Chien et al., Proc. Natl. Acad. Sci. USA, 88: 9578-9582 (1991)) as disclosed by Chevray and Nathans, Proc. Natl.

Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described 30 in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for .beta.-galactosidase. A complete kit (MATCHMAKER.TM.) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Thus, one object of the invention consists of a method for the screening of a candidate substance for its anti-angiogenic activity, wherein said method comprises the steps of:
a) providing a candidate substance; and
b) assaying said candidate substance for its ability to bind to a netrin-1 receptor;

The candidate substances, which may be screened according to the screening method above, may be of any kind, including, without being limited to, natural or synthetic compounds or molecules of biological origin such as polypeptides.

Preferably, the candidate substances that are positively selected at the end of step b) of the screening method above are those which possess the same binding affinity for the netrin-1 receptor than netrin-1, typically human netrin-1. By the “same binding activity”, as applied to candidate substances mimicking natural netrin-1 protein, it is herein intended substances that bind to the netrin-1 receptor with the same order of magnitude than the full length netrin-1, as it can be easily determined by the one skilled in the art, for example by performing any binding assay among those that are generally described above, including two-hybrid or a western blot assay, bio sensor techniques, affinity chromatography, or High Throughput Screening (HTS), that will be described below.

A further object of the invention consists of a method for the screening of a substance that increases angiogenesis, comprising the steps of:

a) providing a candidate substance;

b) assaying said candidate substance for its ability to negatively modulates the netrin-1 receptor activity.

According to a specific embodiment of the method above, at step b) said candidate substance is assayed for its ability to block the binding between netrin-1 and the netrin-1 receptor.

According to another specific embodiment of the method above, at step b) said candidate substance is assayed for its ability to decrease the netrin-1 receptor expression.

Substances that negatively modulate the netrin-1 receptor activity include those substances that prevent the binding of netrin-1 to the netrin-1 receptor. Those potentially pro-angiogenic substances may be screened by assaying for their ability to affect the binding of netrin-1 to the netrin-1 receptor, preferably by carrying out any of the screening methods described therein, including two-hybrid or a westrern blot assay, bio sensor techniques, affinity chromatography, or High Throughput Screening (HTS), that will be described below.

Other substances that negatively modulate the netrin-1 receptor activity are those that decrease or block the netrin-1 receptor expression. One most preferred embodiment of such pro-angiogenic substances of the invention comprise antisense polynucleotide that block translation of the mRNA encoding the netrin-1 receptor. Techniques for measuring a decrease or a complete inhibition of the netrin-1 receptor expression may be any conventional technique used in the art for determining the level of expression of a mRNA, including antibodies directed against the netrin-1 receptor or the use of oligonucleotide probes or primers hybridizing with the mRNA encoding the netrin-1 receptor, including the corresponding probes or primers that are disclosed in the examples herein, and illustratively the oligonucleotides of SEQ ID N^{o} 8-9, 12-13, 14-15, 16-17 or 18-19.

Two Hybrid Screening System

Two-hybrid screening methods are performed for the screening of candidate substances that comprise candidate polypeptides, including mutants or variants of a netrin-1 protein as well as peptide fragments thereof.

In a preferred embodiment, of the screening method, the candidate polypeptide is fused to the LexA binding domain, the netrin-1 receptor protein is fused to Gal 4 activator domain and step (b) is carried out by measuring the expression of a detectable marker gene placed under the control of a LexA regulation sequence that is responsive to the binding of a complete protein containing both the LexA binding domain and the Gal 4 activator domain. For example, the detectable marker gene placed under the control of a LexA regulation sequence can be the β-galactosidase gene or the HIS3 gene, as disclosed in the art.

In a particular embodiment of the screening method, the candidate compound consists of the expression product of a DNA insert contained in a phage vector, such as described by Parmley and Smith (1988). Specifically, random peptide libraries are used. The random DNA inserts encode for peptides of 8 to 20 amino acids in length (Oldenburg et al., 1992, Proc. Natl. Acad. Sci. USA, 85(8): 2444-2448; Valadon et al., 1996, J Mol Biol, 261: 11-22; Lucas, 1994, In: Development and Clinical Uses of Haemophilus b Conjugate; Westerink, 1995, Proc. Natl. Acad. Sci. USA, 92: 4021-4025; Felici et al., 1991, J Mol Biol, 222: 301-310). According to this particular embodiment, the recombinant phages expressing a polypeptide that specifically binds to a netrin-1 receptor protein, preferably the netrin-1 receptor protein of SEQ ID N^{o} 2, are retained as expressing a candidate substance for use in the screening method above.

More precisely, In a first preferred embodiment of the screening method above, the screening system used in step (a) includes the use of a Two-hybrid screening assay. The yeast two-hybrid system is designed to study protein-protein interactions in vivo and relies upon the fusion of a bait protein to the DNA binding domain of the yeast Gal4 protein. This technique is described in the U.S. Pat. No. 5,667,973.

The general procedure of the two-hybrid assay is described hereafter. In an illustrative embodiment, the polynucleotide encoding the netrin-1 receptor is fused to a polynucleotide encoding the DNA binding domain of the Gal4 protein, the fused protein being inserted in a suitable expression vector, for example pAS2 or pM3.

Then, the polynucleotide encoding the candidate polypeptide is fused to a nucleotide sequence in a second expression vector that encodes the activation domain of the Gal4 protein.

The two expression plasmids are transformed into yeast cells and the transformed yeast cells are plated on a selection culture medium which selects for expression of selectable markers on each of the expression vectors as well as GAL4 dependent expression of the HIS3 gene. Transformants capable of growing on medium lacking histidine are screened for gal4 dependent LacZ expression. Those cells which are positive in the histidine selection and the Lac Z assay denote the occurrence of an interaction between the netrin-1 receptor and the candidate polypeptide and allow to quantify the binding of the two protein partners.

Since its original description, the yeast two-hybrid system has beenused extensively to identify protein-protein interactions from many differentorganisms. Simultaneously, a number of variations on a theme based onthe original concept have been described. The original configuration of thetwo-hybrid fusion proteins was modified to expand the range of possibleprotein-protein interactions that could be analyzed. For example,systems were developed to detect trimeric interactions. Finally, the origina Iconcept was turned upside down and ‘reverse n-hybrid systems’ weredeveloped to identify peptides or small molecules that dissociatemacromolecular interactions (Vidal et al., 1999, Yeast forward and reverse ‘n’-hybrid systems. Nucleic Acids Res. 1999 Feb. 15;27(4):919-29). These variations in the two-hybrid system can be applied to the disruption of the interaction between candidates anti-angiogenic polypeptides and a netrin-1 receptor and enters in the scope Is of the present invention.

Western Blot

In another preferred embodiment, of the screening method according to the invention, step (b) consists of subjecting to a gel migration assay the mixture obtained at the end of step (a) and then measuring the binding of the candidate polypeptide with the netrin-1 receptor protein by performing a detection of the complexes formed between the candidate polypeptide and the netrin-1 receptor protein.

The gel migration assay can be carried out by conventional widely used western blot techniques that are well known from the one skilled in the art.

The detection of the complexes formed between the candidate polypeptide and the netrin-1 receptor protein can be easily observed by determining the stain position (protein bands) corresponding to the proteins analysed since the apparent molecular weight of a protein changes if it is in a complex.

On one hand, the stains (protein bands) corresponding to the proteins submitted to the gel migration assay can be detected by specific antibodies for example antibodies specifically directed against the netrin-1 receptor or against the candidate polypeptide, if the latter are available. Alternatively, the candidate polypeptide or the netrin-1 receptor protein can be tagged for an easier revelation of the gel, for example by fusion to GST, HA, poly Histidine chain, or other probes in order to facilitate the identification of the different protein on the gel, according to widely known techniques.

Biosensor

In another preferred embodiment of the screening method above, the screening system used in step (a) includes the use of an optical biosensor such as described by Edwards and Leatherbarrow (1997, Analytical Biochemistry, 246: 1-6) or also by Szabo et al. (1995, Curr. Opinion Struct. Biol., 5(5): 699-705). This technique permits the detection of interactions between molecule in real time, without the need of labelled molecules. This technique is based on the surface plasmon resonance (SPR) phenomenon. Briefly, a first protein partner molecule, for example the candidate polypeptide, is attached to a surface (such as a carboxymethyl dextran matrix). Then, the second protein partner molecule, in this case the netrin-1 receptor, is incubated with the first partner, in the presence or in the absence of the candidate compound to be tested and the binding, including the binding level, or the absence of binding between the first and second protein partner molecules is detected. For this purpose, a light beam is directed towards the side of the surface area of the substrate that does not contain the sample to be tested and is reflected by said surface. The SPR phenomenon causes a decrease in the intensity of the reflected light with a specific combination of angle and wavelength. The binding of the first and second protein partner molecules causes a change in the refraction index on the substrate surface, which change is detected as a change in the SPR signal.

According to the preferred embodiment of the screening method cited above, the “first partner” of the screening system consists of the substrate onto which the first protein partner molecule is immobilised, and the “second partner” of the screening system consists of the second partner protein molecule itself.

Affinity Chromatography

Candidate compounds for use in the screening method above can also be selected by any immunoaffinity chromatography technique using any chromatographic substrate onto which (i) the candidate polypeptide or (ii) the netrin-1 receptor, have previously been immobilised, according to techniques well known from the one skilled in the art.

In a preferred embodiment of the invention, the screening method includes the use of affinity chromatography.

The netrin-1 receptor protein may be attached to a column using conventional techniques including chemical coupling to a suitable column matrix such as agarose, Affi Gel®, or other matrices familiar to those of skill in the art. In some embodiment of this method, the affinity column contains chimeric proteins in which the netrin-1 receptor protein, is fused to glutathion-s-transferase (GST). Then a candidate compound is applied to the affinity column. The amount of the candidate compound retained by the immobilized netrin-1 receptor protein allows measuring the binding ability of said candidate compound on the netrin-1 receptor and thus allows to assess the potential anti-angiogenic activity of said candidate compound.

High Throughput Screening

In another preferred embodiment of the screening method according to the invention, at step (a), the candidate substance and the netrin-1 receptor protein are labelled by a fluorophore. The measurement of the binding of the candidate compound to the netrin-1 receptor protein, at step (b) consists of measuring a fluorescence energy transfer (FRET). Disruption of the interaction by a candidate compound is then followed by decrease or absence of fluorescence transfer. As an exemple, the one skilled in the art can make use of the TRACE technology of fluorescence transfer for Time Resolved Amplified Cryptate Emission developed by Leblanc V, et al. for measuring the FRET. This technology allows to set up very specific and very selective and high throughput screening assays for inhibitors of interaction between the candidate compound and the netrin-1 receptor protein, from which candidate substances will be selected. This technique is based on the transfer of fluorescence from a donor (cryptate) to an acceptor of energy (XL665), when the two molecules are in close proximity in cell extracts.

B. In Vitro and In Vivo Cekk Assays

In a preferred embodiment of the screening method describes above, said method further comprises subsequent steps for assessing the actual anti-angiogenic activity of the candidates substances that are positively selected at the end of step b) above.

Thus, the screening method described above may further comprise the following steps:

c) selecting positively said substance if it binds to the netrin-1 receptor; and

d) assaying the candidate substance positively selected at step c) for its ability to promote angiogenesis in vitro or in vivo.

According to a specific embodiment of the method above, at step d) said candidate substance is assayed for its ability to inhibit filopodial extension of endothelial cells in vitro or in vivo.

In vitro and in vivo assays for assessing the anti-angiogenic activity of a candidate substance are provided in the examples herein.

For instance, the anti-angiogenic activity of said candidate substance may be assayed by assessing its ability at inhibiting endothelial cell migration or at inhibiting filopodial extension of vascular endothelial cells, as shown in the examples.

Compositions capable of Regulating Angiogenesis According to the Invention

The compositions useful in the treatment of angiogenesis associated disorders include, without limitation, antibodies, small organic and inorganic molecules, peptides, phosphopeptides, antisense and ribozyme molecules, triple-helix molecules, etc., that may be positively selected by carrying out any one of the screening methods describes above and that may comprise, depending on the types of screening method employed, either (i) of an anti-angiogenic substance or (ii) of an angiogenesis-promoting substance.

Netrin-1 Proteins and Peptide Fragments Thereof

One kind of substances of therapeutic value according to the invention include the netrin-1 proteins that are disclosed earlier in the present specification, as well as peptide fragment thereof.

Netrin-1 proteins as well as peptide fragments of netrin-1 proteins that retain the anti-angiogenesis activity of netrin-1 can be used for inhibiting angiogenesis, according to the invention.

Further, netrin-1 peptide fragments that bind to the netrin-1 receptor, but does not retain the anti-angiogenesis activity of netrin-1, prevent the binding of netrin-1 to its corresponding receptor and thus can be used for promoting angiogenesis, according to the invention.

In a preferred embodiment, an anti-angiogenic substance of the invention comprises a polypeptide having at least 80% amino acid identity with a mammal netrin-1 protein, most preferably a polypeptide having at least 80% amino acid identity with a mammalian netrin-1 protein selected from the group consisting of SEQ ID N^{o} 1 to SEQ ID N^{o} 4.

As already specified, the anti-angiogenic substance may comprise a polyeptide having at least 81%, 82%, 83%, 84%, 85%, 86%, 87%, 88%, 89%, 90%, 91%, 92%, 93%, 94%, 95%, 96%, 97%, 98% or 99% amino acid identity with a mammalian netrin-1 protein, and most preferably with a mammalian netrin-1 protein selected from the group consisting of SEQ ID N^{o}1 to SEQ ID N^{o} 4.

Alternatively, the anti-angiogenic substance may comprise a mammalian netrin-1 protein, and most preferably with a mammalian netrin-1 protein selected from the group consisting of SEQ ID N^{o}1 to SEQ ID N^{o} 4.

In another preferred embodiment, an anti-angiogenic substance according to the invention comprises a peptide fragment of a mammalian netrin-1 protein that still binds to the corresponding netrin-1 receptor, most preferably with a binding affinity of the same order of magnitude as that of the full length netrin-1 protein of interest.

In certain preferred embodiments, useful peptide fragments of a mammal nerin-1 protein comprise at least 400 consecutive amino acids of a native netrin-1 protein. Most preferably, useful peptide fragments comprise at least 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498; 499, 500, 501, 502, 503, 504, 505, 506, 507, 508, 509, 510, 511, 512, 513, 514, 515, 516, 517, 518, 519, 420, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598; 599, 600 or 601 consecutive amino acids of a mammal netrin-1 protein, most preferably of a mammal netrin-1 protein selected from the group consisting of SEQ ID N^{o} 1 to 4.

In other specific embodiments, useful peptide fragments of a mammal nerin-1 protein comprise at least a portion of at least 200 consecutive nucleotides of the C-terminal part of netrin-1. Are encompassed in these other specific embodiments peptide fragments of at least 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 139, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298; 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398; 399 or 400 consecutive nucleotides of the C-terminal portion of a netrin-1. Preferably, the C-terminal amino acid of said peptide fragments corresponds to an amino acid of the netrin-1 of reference located in the last 50, alternatively in the last 30, alternatively in the last 20, alternatively in the last 20; alternatively in the last 10 C-terminal amino acids of said netrin-1 protein of reference, including the netrin-1 proteins of SEQ ID N^{o} 1-4 or any variant thereof.

In a further embodiment, a pro-angiogenic substance according to the invention may encompass netrin-1 peptide fragments of a shorter amino acid length than those that are potentially anti-angiogenic.

Preferably, pro-angiogenic netrin-1 peptide fragments comprises a polypeptide comprising at most 100 consecutive amino acids from a mammal netrin-1 protein, most preferably from a mammal naterin-1 protein selected from the group consisting of SEQ ID N^{o}1 to SEQ ID N^{o}4.

Preferably, a pro-angiogenic netrin-1 peptide fragment comprises less than 50 consecutive amino acids from a mammal netrin-1 protein, most preferably from a mammal naterin-1 protein selected from the group consisting of SEQ ID N^{o}1 to SEQ ID N^{o}4 .

Preferably a pro-angiogenic netrin-1 peptide fragment comprises at least 10 consecutive aminoacids from a mammal netrin-1 protein, most preferably from a mammal naterin-1 protein selected from the group consisting of SEQ ID N^{o}1 to SEQ ID N^{o}4.

Compositions Comprising Netrin-1 Receptor or a Peptide Fragment Thereof

A further embodiment of a pro-angiogenic substance comprises a netrin-1 receptor, a soluble form of the netrin-1 receptor, or a peptide fragment thereof for which the binding ability to netrin-1 has been retained.

Preferably, the soluble form of the netrin-1 receptor or a fragment thereof comprises at least a portion of the extracellular domain of the full length netrin-1 receptor. The complete extracellular domain of the full length netrin-1 receptor comprises the Tsp1, Tsp2, Ig1 and 1g2 domains of said netrin-1 receptor.

According to certain embodiments of soluble forms of a netrin-1 receptor, a soluble form of netrin-1 receptor encompasses the soluble forms that comprise the Ig1 domain, the 1g2 domain or both the 1g1 and 1g2 domains of said netrin-1 receptor. According to these embodiments, soluble forms of netrin-1 receptor encompass polypeptides that comprise the N-terminal portion of said netrin-1 receptor that includes the 1g2 domain or that includes both the 1g2 and the 1g1 domains. According to these embodiments, soluble forms of a netrin-1 receptor encompass polypeptides having at least 100 consecutive amino acids of the N-terminal portion of said netrin-1 receptor, alternatively at least 150 consecutive amino acids of said N-terminal portion, alternatively at least 200 consecutive amino acids of said N-terminal portion, alternatively at least 250 consecutive amino acids of said N-terminal portion, alternatively at least 300 consecutive amino acids of said N-terminal portion of said netrin-1 receptor. Preferably, the N-terminal portion of said peptide fragments corresponds to an amino acid of the netrin-1 receptor of reference located in the first 10, alternatively in the first 20, alternatively in the first 30, alternatively in the first 40, alternatively in the first 50, alternatively in the first 60 N-terminal amino acids of said netrin-1 receptor of reference, including the netrin-1 receptor of SEQ ID N^{o} 5. or any variant therof.

Preferably, the netrin-1 receptor of the invention comprises the polypeptide of SEQ ID N^{o}5.

Such pro-angiogenic polypeptide may also comprise a fusion protein comprising a netrin-1 receptor and any heterologous polypeptide.

For example, an illustrative embodiment of such a pro-angiogenic substance according to the invention is given in the examples, under the form of a fusion protein between human netrin-1 receptor and an Fc fragment of an immunoglobulin.

Production of the Anti-Angiogenic or of the Pro-angiogenic Polypeptides The description below relates primarily to production of the anti-angiogenic or the pro-angiogenic polypeptides according to the invention by culturing cells transformed or transfected with a vector containing nucleic acid encoding corresponding polypeptides. It is, of course, contemplated that alternative methods that are well known in the art may be employed to prepare the anti-angiogenic or the pro-angiogenic polypeptides of interest according to the invention. For instance, the polypeptide sequence of interest, or portions thereof, may be produced by direct peptide synthesis using solid-phase techniques. See, e.g., Stewart et al., Solid-Phase Peptide Synthesis (W. H. Freeman Co.: San Francisco, Calif., 1969); Merrifield, J. Am. Chem. Soc., 85: 2149-2154 (1963). In vitro protein synthesis may be performed using manual techniques or by automation. Automated synthesis may be accomplished, for instance, with an Applied Biosystems Peptide Synthesizer (Foster City, Calif.) using manufacturer's instructions. Various portions of the polypeptide of interest may be chemically synthesized separately and combined using chemical or enzymatic methods to produce the full-length polypeptide of interest

Isolation of DNA Encoding Anti-Angiogenic or the Pro-Angiogenic Polypeptides of Interest

DNA encoding the polypeptide of interest may be obtained from a cDNA library prepared from tissue believed to possess the mRNA encoding it and to express it at a detectable level. Accordingly, DNAs encoding the netrin-1 polypeptides or the netrin-1 receptor polypeptides can be conveniently obtained from cDNA libraries prepared from human tissues.

Selection and Transformation of Host Cells

Host cells are transfected or transformed with expression or cloning vectors described herein for polypeptide of interest production and cultured in conventional nutrient media modified as appropriate for inducing promoters, selecting transformants, or amplifying the genes encoding the desired sequences. The culture conditions, such as media, temperature, pH, and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991).

Methods of transfection are known to the ordinarily skilled artisan, for example, CaPO.sub.4 treatment and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene 23: 315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transformations have been described in U.S. Pat. No. 4,399,216. Transformations into yeast are typically carried out according to the method of Van Solingen et al., J. Bact., 130: 946 (1977) and Hsiao et al., Proc. Natl. Acad. Sci. (USA), 76: 3829 (1979). However, other methods for introducing DNA into cells, such as by nuclear microinjection, electroporation, bacterial protoplast fusion with intact cells, or polycations, e.g., polybrene or polyornithine, may also be used. For various techniques for transforming mammalian cells, see, Keown et al., Methods in Enzymology, 185: 527-537 (1990) and Mansour et al., Nature, 336: 348-352 (1988).

Suitable host cells for cloning or expressing the DNA in the vectors herein include prokaryote, yeast, or higher eukaryote cells. Suitable prokaryotes include, but are not limited to, eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325); and K5772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan.sup.r; E. coli W3110 strain 37D6, which has the complete genotype tona ptr3 phoA E15 (argF-lac)169 degp ompT rbs7 ilvG kan.sup.r; E. coli W3110 strain 40B4, which is strain 37D6 with a non-kanamycin resistant degp deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for vectors encoding the anti-angiogenic or the pro-angiogenic polypeptide. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9: 968-975 (1991)) such as, e.g., K. lactis (MW98-8C, CBS683, CBS4574; Louvencourt et al., J. Bacteriol., 737 [1983]), K. fragilis (ATCC 12,424), K. bulgaricus (ATCC 16,045), K. wickeramii(ATCC 24,178), K. waltii(ATCC 56,500), K. drosophilarum(ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28: 265-278 [1988]); Candida; Trichoderma reesia (EP 244,234); Neurospora crassa (Case et al., Proc. Natl. Acad. Sci. USA, 76: 5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112: 284-289 [1983]; Tilburn et al., Gene, 26: 205-221 [1983]; Yelton et al., Proc. Natl. Acad. Sci. USA, 81: 1470-1474 [1984]) and A. niger (Kelly and Hynes, EMBO J., 4: 475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of nucleic acid encoding glycosylated polypeptides of interest are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen. Virol., 36: 59 (1977)); Chinese hamster ovary cells/-DHFR(CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

Selection and Use of a Replicable Vector

The nucleic acid (e.g., cDNA or genomic DNA) encoding the polypeptide of interest may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence if the sequence is to be secreted, an origin of replication, one or more marker genes, an enhancer element, a promoter, and a transcription termination sequence. Construction of suitable vectors containing one or more of these components employs standard ligation techniques that are known to the skilled artisan.

The polypeptide of interest may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the DNA encoding the polypeptide of interest that is inserted into the vector. The signal sequence may be a prokaryotic signal sequence selected, for example, from the group of the alkaline phosphatase, penicillinase, Ipp, or heat-stable enterotoxin 11 leaders. For yeast secretion the signal sequence may be, e.g., the yeast invertase leader, alpha factor leader (including Saccharomyces and Kluyveromyces .alpha.-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2.mu. plasmid origin is suitable for yeast, and various viral origins (SV40, polyoma, adenovirus, VSV, or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the nucleic acid encoding the anti-angiogenic or the pro-angiogenic polypeptide of interest such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77: 4216 (1980). A suitable selection gene for use in yeast is the trp 1 gene present in the yeast plasmid YRp7. Stinchcomb et al., Nature, 282: 39 (1979); Kingsman et al., Gene, 7: 141 (1979); Tschemper et al, Gene, 10: 157 (1980). The trpl gene provides a selection marker for a mutant strain of yeast lacking the ability to grow in tryptophan, for example, ATCC No. 44076 or PEP4-1. Jones, Genetics, 85: 12 (1977).

Expression and cloning vectors usually contain a promoter operably linked to the nucleic acid sequence encoding the anti-angiogenic or the pro-angiogenic polypeptide to direct mRNA synthesis. Promoters recognized by a variety of potential host cells are well known. Promoters suitable for use with prokaryotic hosts include the beta.-lactamase and lactose promoter systems (Chang et al., Nature, 275: 615 (1978); Goeddel et al., Nature, 281: 544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80: 21-25 (1983)). promoters for use in bacterial systems also will contain a Shine-Dalgarno (S.D.) sequence operably linked to the DNA encoding the anti-angiogenic or the pro-angiogenic polypeptide of interest.

Examples of suitable promoting sequences for use with yeast hosts include the promoters for 3-phosphoglycerate kinase (Hitzeman et al., J. Biol. Chem., 255: 2073 (1980)) or other glycolytic enzymes (Hess et al., J. Adv. Enzyme Reg., 7: 149 (1968); Holland, Biochemistry, 17: 4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters that are inducible promoters having the additional advantage of transcription controlled by growth conditions are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Nucleic acid of interest transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus, and Simian Virus 40 (SV40); by heterologous mammalian promoters, e.g., the actin promoter or an immunoglobulin promoter; and by heat-shock promoters, provided such promoters are compatible with the host cell systems.

Transcription of a DNA encoding the polypeptide of interest by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, .alpha.-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the sequence coding for polypeptides of interest, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the anti-angiogenic or the pro-angiogenic polypeptide of interest.

Still other methods, vectors, and host cells suitable for adaptation to the synthesis of the anti-angiogenic or the pro-angiogenic polypeptide of interest in recombinant vertebrate cell culture are described in Gething et al., Nature, 293: 620-625 (1981); Mantei et al., Nature, 281: 40-46 (1979); EP 117,060; and EP 117,058.

Purification of the polypeptides of interest

Forms of polypeptides of interest may be recovered from culture medium or from host cell lysates. If membrane-bound, it can be released from the membrane using a suitable detergent solution (e.g., TRITON—X.TM. 100) or by enzymatic cleavage. Cells employed in expression of nucleic acid encoding the anti-angiogenic or the pro-angiogenic polypeptide of interest can be disrupted by various physical or chemical means, such as freeze-thaw cycling, sonication, mechanical disruption, or cell-lysing agents. It may be desired to purify the polypeptide of interest from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; Protein A Sepharose columns to remove contaminants such as IgG; and metal chelating columns to bind epitope-tagged forms of the anti-angiogenic or the pro-angiogenic polypeptide of interest. Various methods of protein purification may be employed and such methods are known in the art and described, for example, in Deutscher, Methods in Enzymology, 182 (1990); Scopes, Protein Purification: Principles and Practice (Springer-Verlag: New York, 1982). The purification step(s) selected will depend, for example, on the nature of the production process used and the particular polypeptide produced.

Finally, specific embodiments for obtaining a nucleic acid encoding human netrin-1, inserting said nucleic acid in a suitable expression vector, and transfecting host cells with said vector in order to produce the human netrin-1 protein are disclosed in the U.S. Pat. No. 6,218,526.

Further, specific embodiments for obtaining a nucleic acid encoding netrin-1 receptors, inserting said nucleic acid in a suitable expression vector, and transfecting host cells with said vector in order to produce human netrin-1 receptors are disclosed in the PCT application n^{o} WO 98/37085.

Antibodies

One kind of anti- or pro-angiogenic substances of interest according to the present invention comprise antibodies.

Antibodies that are useful as anti-angiogenic substances according to the invention encompass those antibodies that mimic the binding of netrin-1 to the netrin-1 receptor. Preferably, the antibodies useful as anti-angiogenic substances include anti-netrin-1 receptor agonist antibodies capable of eliciting, enhancing, or upregualting netrin-1 induced activity of netrin-1 receptor.

Antibodies that are useful as pro-angiogenic substances according to the invention encompass those antibodies that bind to netrin-1 and prevent the. binding of netrin-1 on the netrin-1 receptor. Antibodies that are useful as pro-angiogenic substances according to the invention may also encompass those antibodies that bind to the netrin-1 receptor and do not mimic the binding of netrin-1 on the netrin-1 receptor, while preventing the binding of netrin-1 on the netrin-1 receptor.

Description is given below of antibodies as angiogenesis modulating substances according to the invention in reference to the anti-angiogenic antibodies that bind to netrin-1 receptor and agonizes netrin-1 induced activity of netrin-1 receptor. However, same description of antibodies and methods for obtaining antibodies may be used for the pro-angiogenic antibodies that are described above.

More specific examples of potential anti-angiogenic substances include an antibody that binds to the fusions of immunoglobulin with a netrin-1 receptor polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential anti-angiogenic substance may be a closely related protein, for example, a mutated form of a netrin-1 polypeptide that recognizes the netrin-1 receptor but imparts no effect, thereby competitively inhibiting the action of the polypeptide.

For the purpose of the present invention, the antibodies that are described hereunder are selected from the group consisting of antibodies directed against netrin-1 and antibodies directed against the netrin-1 receptor.

As already explained above, the same techniques are used for obtaining or manufacturing pro-angiogenic antibodies of the invention.

Some of the most promising drug candidates according to the present invention are antibodies and antibody fragments that may inhibit the production or the gene product of the genes identified herein and/or reduce the activity of the gene products.

Definitions of Relevance Pertaining to Antibodies Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. The term “antibody” is used in the broadest sense and specifically covers, without limitation, intact monoclonal antibodies, polyclonal antibodies, multispecific antibodies (e.g., bispecific antibodies) formed from at least two intact antibodies, and antibody fragments, so long as they exhibit the desired biological activity.

“Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V.sub.H) followed by a number of constant domains. Each light chain has a variable domain at one end (V.sub.L) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “variable” refers to the fact that certain portions of the variable domains differ extensively in sequence among antibodies and are used in the binding and specificity of each particular antibody to and for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework regions (FR). The variable domains of native heavy and light chains each comprise four FR regions, largely adopting a beta.-sheet configuration, connected by three CDRs, which form loops connecting, and in some cases forming part of, the .beta.-sheet structure. The CDRs in each chain are held together in close proximity by the FR regions and, with the CDRs from the other chain, contribute to the formation of the antigen-binding site of antibodies. See, Kabat et al., NIH Publ. No. 91-3242, Vol. 1, pages 647-669 (1991). The constant domains are not involved directly in binding an antibody to an antigen, but exhibit various effector functions, such as participation of the antibody in antibody-dependent cellular toxicity.

“Antibody fragments” comprise a portion of an intact antibody, preferably the antigen-binding or variable region of the intact antibody. Examples of antibody fragments include Fab, Fab′, F(ab′).sub.2, and Fv fragments; diabodies; linear antibodies (Zapata et al., Protein Eng., 8(10): 1057-1062 (1995)); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′).sub.2 fragment that has two antigen-combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V.sub.H-V.sub.Ldimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′).sub.2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, Is called kappa (.kappa.) and lambda (.lambda.), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM; and several of these may be further divided into subclasses (isotypes), e.g., IgG1, IgG2, IgG3, IgG4, IgA, and IgA2. The heavy-chain constant domains that correspond to the different classes of immunoglobulins are called alpha., delta., epsilon., .gamma., and mu., respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352: 624-628 (1991) and Marks et al., J. Mol. Biol, 222: 581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al., Proc. Nati. Acad. Sci. USA, 81: 6851-6855 (1984).

“Humanized” forms of non-human (e.g., murine) antibodies are chimeric immunoglobulins, immunoglobulin chains, or fragments thereof (such as Fv, Fab, Fab′, F(ab′).sub.2, or other antigen-binding subsequences of antibodies) that contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat or rabbit having the s desired specificity, affinity, and capacity. In some instances, Fv FR residues of the human immunoglobulin are replaced by corresponding non-human residues. Furthermore, humanized antibodies may comprise residues that are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody preferably also will comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992). The humanized antibody includes a PRIMATIZED.TM. antibody wherein the antigen-binding region of the antibody is derived from an antibody produced by immunizing macaque monkeys with the antigen of interest.

“Single-chain Fv” or “sFv” antibody fragments comprise the V.sub.H and V.sub.L domains of an antibody, wherein these domains are present in a single polypeptide chain. Preferably, the Fv polypeptide further comprises a polypeptide linker between the V.sub.H and V.sub.L domains that enables the sFv to form the desired structure for antigen binding. For a review of sFv see, Pluckthun in The Pharmacology of Monoclonal Antibodies, Vol. 113, Rosenburg and Moore, eds. (Springer-Verlag: New York, 1994), pp. 269-315.

The term “diabodies” refers to small antibody fragments with two antigen-binding sites, which fragments comprise a heavy-chain variable domain (V.sub.H) connected to a light-chain variable domain (V.sub.L) in the same polypeptide chain (V.sub.H-V.sub.L). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90: 6444-6448 (1993).

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells, since at least one component of the antibody's natural environment will not be present. Ordinarily, however, isolated antibody will be prepared by at least one purification step.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The word “label” when used herein refers to a detectable compound or other composition that is conjugated directly or indirectly to the antibody so as to generate a “labeled” antibody. The label may be detectable by itself (e.g., radioisotope labels or fluorescent labels) or, in the case of an enzymatic label, may catalyze chemical alteration of a substrate compound or composition that is detectable. Radionuclides that can serve as detectable labels include, for example, 1-131, 1-123, 1-125, Y-90, Re-188, At-211, Cu-67, Bi-212, and Pd-109. The label may also be a non-detectable entity such as a toxin.

By “solid phase” is meant a non-aqueous matrix to which an antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant that is useful for delivery of a drug (such as the netrin-1 polypeptide or antibodies directed against the netrin-1 receptor disclosed herein) to a mammal. The components of the liposome are commonly arranged in a bilayer formation, similar to the lipid arrangement of biological membranes.

As used herein, the term “immunoadhesin” designates antibody-like molecules that combine the binding specificity of a heterologous protein (an “adhesin”) with the effector functions of immunoglobulin constant domains. Structurally, the immunoadhesins comprise a fusion of an amino acid sequence with the desired binding specificity that is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD, or IgM.

i. Polyclonal Antibodies

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the netrin-1 receptor polypeptide or a fusion protein thereof. It may be useful to conjugate the immunizing agent to a protein known to be immunogenic in the mammal being immunized. Examples of such immunogenic proteins include, but are not limited to, keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A or synthetic trehalose dicorynomycolate). The immunization protocol may be selected by one skilled in the art without undue experimentation.

ii. Monoclonal Antibodies

The anti-netrin-1 receptor antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the netrin-1 receptor polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice (New York: Academic Press, 1986), pp. 59-103. Immortalized cell lines are usually transformed mammalian cells, particularly myeloma cells of rodent, bovine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high-level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. Kozbor, J. Immunol., 133:3001 (1984); Brodeur et al., Monoclonal Antibody Production Techniques and Applications (Marcel Dekker, Inc.: New York, 1987) pp.51-63.

The culture medium in which the hybridoma cells are cultured can then be assayed for the presence of monoclonal antibodies directed against the netrin-1 receptor polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods. Goding, supra. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, Protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy- and light-chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using routine techniques known in the art.

iii. Human and Humanized Antibodies

The anti-netrin-1 receptor antibodies may further comprise humanized antibodies or human antibodies.

Humanized forms of non-human (e.g., murine) antibodies are chimeric immunoglobulins, immunoglobulin chains, or fragments thereof (such as Fv, Fab, Fab′, F(ab′).sub.2 or other antigen-binding subsequences of antibodies) that contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody preferably also will comprise at least a portion of an immunoglobulin constant region (Fc), typically that of a human immunoglobulin. Jones et al., Nature, 321: 522-525 (1986); Riechmann et al., Nature, 332: 323-329 (1988); Presta, Curr. Op. Struct. Biol., 2:593-596 (1992).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries. Hoogenboom and Winter, J. Mol. Biol., 227: 381(1991); Marks et al., J. Mol. Biol., 222: 581(1991). The techniques of Cole et al. and Boemer et al. are also available for the preparation of human monoclonal antibodies. Cole et al., Monoclonal Antibodies and Cancer Therapy, Alan R. Liss, p. 77 (1985) and Boemer et al., J. Immunol., 147(1): 86-95 (1991). Similarly, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and in the following scientific publications: Marks et al., BiofTechnology, 10: 779783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

iv. Bispecific Antibodies

Bispecific antibodies are monoclonal, preferably human or humanized, antibodies that have binding specificities for at least two different antigens. In the present case, one of the binding specificities is for the netrin-1 receptor polypeptide, the other one is for any other antigen, and preferably for a cell-surface protein or receptor or receptor subunit.

Methods for making bispecific antibodies are known in the art. Traditionally, the recombinant production of bispecific antibodies is based s on the co-expression of two immunoglobulin heavy-chain/light-chain pairs, where the two heavy chains have different specificities. Milstein and Cuello, Nature, 305: 537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10: 3655-3659 (1991).

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH 1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see, for example, Suresh et al., Methods in Enzymology, 121: 210 (1986).

v. Heteroconiugate Antibodies

Heteroconjugate antibodies are composed of two covalently joined antibodies. Such antibodies have, for example, been proposed to target immune-system cells to unwanted cells (U.S. Pat. No. 4,676,980), and for treatment of HIV infection. WO 91/00360; WO 92/200373; EP 03089. It is contemplated that the antibodies may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980.

vi. Effector Function Engineering

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See, Caron et al., J. Exp. Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al., Cancer Research, 53: 2560-2565 (1993). Alternatively, an antibody can be engineered that has dual Fc regions and may thereby have enhanced complement lysis and ADCC capabilities. See, Stevenson et al., Anti-Cancer Drug Design. 3: 219-230 (1989).

vii. Immunoconiugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof, or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include .sup.212Bi, .sup.1311, .sup.131 In, .sup.90Y, and .sup.186Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCI), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, WO94/11026.

In another embodiment, the antibody may be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

viii. Immunoliposomes

The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation timeare disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See, Gabizon et al., J. National Cancer Inst., 81(19): 1484 (1989).

ix. Pharmaceutical Compositions of Antibodies

Antibodies specifically binding a netrin-1 receptor polypeptide identified herein, as well as other molecules identified by the screening assays disclosed hereinbefore, can be administered for the treatment of various disorders as noted above and below in the form of pharmaceutical compositions.

x. Methods of Treatment using the Antibody

It is contemplated that the antibodies to a netrin-1 receptor polypeptide that mimick the binding of netrin-1 to the netrin-1 receptor may be used to prevent or to treat various conditions or diseases associated with undesirable neovascularization.

It is also contemplated that the antibodies that prevent the binding between netrin-1 and the netrin-1 receptor, which are most preferably antibodies that bind to netrin-1, may be used to prevent or treat conditions or diseases wherein angiogenesis has to be promoted.

The antibodies are administered to a mammal, preferably a human, in agreement with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Intravenous administration of the antibody is preferred.

Other therapeutic regimens may be combined with the administration of the antibodies of the instant invention as noted above. For example, if the antibodies are to treat cancer, the patient to be treated with such antibodies may also receive radiation therapy. Alternatively, or in addition, a chemotherapeutic agent may be administered to the patient. Preparation and dosing schedules for such chemotherapeutic agents may be used according to manufacturers' instructions or as determined empirically by the skilled practitioner. Preparation and dosing schedules for such chemotherapy are also described in Chemotherapy Service, Ed., M. C. Perry (Williams & Wilkins: Baltimore, Md., 1992). The chemotherapeutic agent may precede, or follow administration of the antibody, or may be given simultaneously therewith. The antibody may be combined with an anti-estrogen compound such as tamoxifen or EVISTA.TM. or an anti-progesterone such as onapristone (see, EP 616812) in dosages known for such molecules.

If the antibodies are used for treating cancer, it may be desirable also to administer antibodies against other tumor-associated antigens, such as antibodies that bind to one or more of the ErbB2, EGFR, ErbB3, ErbB4, or VEGF receptor(s). These also include the agents set forth above. Also, the antibody is suitably administered serially or in combination with radiological treatments, whether involving irradiation or administration of radioactive substances. Alternatively, or in addition, two or more antibodies binding the same or two or more different antigens disclosed herein may be co-administered to the patient. Sometimes, it may be beneficial also to administer one or more cytokines to the patient.

In a preferred embodiment, the antibodies herein are co-administered with a growth-inhibitory agent. For example, the growth-inhibitory agent may be administered first, followed by an antibody of the present invention. However, simultaneous administration or administration of the antibody of the present invention first is also contemplated. Suitable dosages for the growth-inhibitory agent are those presently used and may be lowered due to the combined action (synergy) of the growth-inhibitory agent and the antibody herein.

In one embodiment, vascularization of tumors is attacked in 20 combination therapy. The anti-netrin-1 receptor antibody and another antibody (e.g., anti-VEGF) are administered to tumor-bearing patients at therapeutically effective doses as determined, for example, by observing necrosis of the tumor or its metastatic foci, if any. This therapy is continued until such time as no further beneficial effect is observed or clinical examination shows no trace of the tumor or any metastatic foci. The anti-netrin-1 receptor agonist antibody can also be used in combination with other anti-tumor agents, such as alpha-, beta-, or gamma-interferon, anti-HER2 antibody, heregulin, anti-heregulin antibody, D-factor, interleukin-1 (IL-1), interleukin-2 (IL-2), granulocyte-macrophage colony stimulating factor (GM-CSF), or agents that promote microvascular coagulation in tumors, such as anti-Protein C antibody, anti-Protein S antibody, or C4b binding protein (see, WO 91/01753, published 21 Feb. 1991), or heat or radiation.

In other embodiments, a FGF or PDGF antagonist, such as an anti-FGF or an anti-PDGF neutralizing antibody, is administered to the patient in conjunction with the anti-netrin-1 receptor polypeptide antibody. Treatment with anti-netrin-1 receptor polypeptide antibodies preferably may be suspended during periods of wound healing or desirable neovascularization.

For the prevention or treatment of angiogenic disorder, the appropriate dosage of an antibody herein will depend on the type of disorder to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

For example, depending on the type and severity of the disorder, about 1.mu.g/kg to 50 mg/kg (e.g., 0.1-20 mg/kg) of antibody is an initial candidate dosage for administration to the patient, whether, for example, by one or more separate administrations, or by continuous infusion. A typical daily or weekly dosage might range from about 1.mu.g/kg to 100 mg/kg or more, depending on the factors mentioned above. For repeated administrations over several days or longer, depending on the condition, the treatment is repeated or sustained until a desired suppression of disorder symptoms occurs. However, other dosage regimens may be useful. The progress of this therapy is easily monitored by conventional techniques and assays, including, for example, radiographic tumor imaging.

Antisense Polynucleotides

Other potential pro-angiogenic substances according to the invention are those that decrease the netrin-1 receptor expression.

Another potential pro-angiogenic substance is an antisense RNA or DNA construct prepared using antisense technology, where, e.g., an antisense RNA or DNA molecule acts to block directly the translation of mRNA by hybridizing to targeted mRNA and preventing protein translation.

More precisely, is encompassed as a pro-angiogenic substance according to the present invention any antisense nucleic acid, including any antisense RNA or DNA molecule, that will finally prevent the binding between netrin-1 and the netrin-1 receptor. Those antisense nucleic acids encompass (i) the antisense nucleic acids that block translation of mRNA encoding netrin-1 and (ii) the antisense nucleic acids that block translation of mRNA encoding the netrin-1 receptor.

Thus, according to a further embodiment of a pro-angiogenic substance of the invention, said pro-angiogenic substance comprises a netrin-1 receptor-specific antisense polynucleotide. Said antisense polynucleotide blocks translation of the mRNA encoding the netrin-1 receptor.

According to a still further embodiment of a pro-angiogenic substance of the invention, said pro-angiogenic substance comprises a netrin-1 -specific antisense polynucleotide. Said antisense polynucleotide blocks translation of the mRNA encoding netrin-1.

Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature netrin-1 receptor, is used to design an antisense RNA oligonucleotide of from about 10 to 40 base pairs in length. A DNA oligonucleotide is designed to be complementary to a region of the gene involved in transcription (triple helix--see, Lee et al., Nucl. Acids Res., 6:3073 (1979); Cooney et al., Science, 241: 456 (1988); Dervan et al., Science, 251:1360 (1991)), thereby preventing transcription and the production of the netrin-1 receptor. The antisense RNA oligonucleotide hybridizes to the mRNA in vivo and blocks translation of the mRNA molecule into the netrin-1 receptor protein (antisense--Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the netrin-1 receptor. When antisense DNA is used, oligodeoxyribonucleotides derived from the translation-initiation site, e.g., between about −10 and +10 positions of the target gene nucleotide sequence, are preferred.

Similarly, as already described, the same techniques are carried out for designing and using antisense nucleic acids that block translation of the mRNA encoding the netrin-1 protein.

Antisense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

Illustrative embodiments of useful antisense polynucleotides are provided in the examples herein.

For instance, an antisense polynucleotide that blocks translation of the mRNA encoding a netrin-1 protein may comprise the antisense nucleic acid of SEQ ID No 6.

For instance, an antisense polynucleotide that blocks translation of the mRNA encoding a netrin-1 receptor may comprise the antisense nucleic acid of SEQ ID N^{o} 7.

Pharmaceutical Methods According to the Invention and Compositions for Carrying Out These Methods.

The various anti-angiogenic substances and the various pro-angiogenic substances that are disclosed therein are pharmaceutically useful as a prophylactic and therapeutic agent for various disorders and diseases as set forth above.

Thus, this invention further relates to a pharmaceutical composition for preventing or treating a condition or a disease linked to a strong angiogenesis comprising an anti-angiogenic substance that is described herein, including an anti-angiogenic substance that has been selected through any one of the methods for screening a candidate substance for its anti-angiogenic activity that are disclosed earlier in the present specification.

According to a specific embodiment of such an anti-angiogenic pharmaceutical composition, said pharmaceutical composition further comprises an effective amount of a second substance selected from the group of an anti-angiogenic substance and an anti-cancer substance.

The present invention still further relates to a pharmaceutical composition for preventing or treating a condition or a disease linked to a defect in angiogenesis comprising a pro-angiogenic substance that is described herein, including a pro-angiogenic substance that has been selected through any one of the methods for screening a candidate substance for its pro-angiogenic activity that are disclosed earlier in the present specification.

Therapeutic compositions of the anti-angiogenic or the pro-angiogenic substances are prepared for storage by mixing the desired molecule having the appropriate degree of purity with optional pharmaceutically acceptable carriers, excipients, or stabilizers (Remineton's Pharmaceutical Sciences, 16th edition, Osol, A. ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN.TM., PLURONICS.TM. or polyethylene glycol (PEG).

Additional examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of the anti-angiogenic or of the pro-angiogenic substances according to the invention include polysaccharides such as sodium carboxymethylcellulose or methylcellulose, polyvinylpyrrolidone, polyacrylates, polyoxyethylene-polyoxypropylene-blo-ck polymers, polyethylene glycol, and wood wax alcohols. For all administrations, conventional depot forms are suitably used. Such forms include, for example, microcapsules, nano-capsules, s liposomes, plasters, inhalation forms, nose sprays, sublingual tablets, and sustained-release preparations. The anti-angiogenic or the pro-angiogenic substances according to the invention will typically be formulated in such vehicles at a concentration of about 0.1 mg/ml to 100 mg/ml.

Another formulation comprises incorporating anti-angiogenic or a pro-angiogenic substance according to the invention into formed articles. Such articles can be used in modulating endothelial cell growth and angiogenesis. In addition, tumor invasion and metastasis may be modulated with these articles.

The anti-angiogenic or the pro-angiogenic substances to be used for in vivo administration must be sterile. This is readily accomplished by filtration through sterile filtration membranes, prior to or following lyophilization and reconstitution. The anti-angiogenic or the pro-angiogenic substance of interest ordinarily will be stored in lyophilized form or in solution if administered systemically. If in lyophilized form, the anti-angiogenic or the pro-angiogenic substance of interest is typically formulated in combination with other ingredients for reconstitution with an appropriate diluent at the time for use. An example of a liquid formulation of a anti-angiogenic or the pro-angiogenic substance of interest is a sterile, clear, colorless unpreserved solution filled in a single-dose vial for subcutaneous injection. Preserved pharmaceutical compositions suitable for repeated use may contain, for example, depending mainly on the indication and type of polypeptide:

a) an anti-angiogenic or the pro-angiogenic substance of interest;

b) a buffer capable of maintaining the pH in a range of maximum stability of the polypeptide or other molecule in solution, preferably about 4-8;

c) a detergent/surfactant primarily to stabilize the polypeptide or molecule against agitation-induced aggregation;

d) an isotonifier;

e) a preservative selected from the group of phenol, benzyl alcohol and a benzethonium halide, e.g., chloride; and

f) water.

If the detergent employed is non-ionic, it may, for example, be polysorbates (e.g., POLYSORBATE.TM. (TWEEN.TM.) 20,80, etc.) or poloxamers (e.g., POLOXAMER.TM. 188). The use of non-ionic surfactants permits the formulation to be exposed to shear surface stresses without causing denaturation of the polypeptide. Further, such surfactant-containing formulations may be employed in aerosol devices such as those used in a pulmonary dosing, and needleless jet injector guns (see, e.g., EP 257,956).

An isotonifier may be present to ensure isotonicity of a liquid composition of the anti-angiogenic or the pro-angiogenic substance of interest, and includes polyhydric sugar alcohols, preferably trihydric or higher sugar alcohols, such as glycerin, erythritol, arabitol, xylitol, sorbitol, and mannitol. These sugar alcohols can be used alone or in combination. Alternatively, sodium chloride or other appropriate inorganic salts may be used to render the solutions isotonic.

The buffer may, for example, be an acetate, citrate, succinate, or phosphate buffer depending on the pH desired. The pH of one type of liquid formulation of this invention is buffered in the range of about 4 to 8, preferably about physiological pH.

The preservatives phenol, benzyl alcohol and benzethonium halides, e.g., chloride, are known antimicrobial agents that may be employed.

Therapeutic anti-angiogenic or pro-angiogenic substances compositions generally are placed into a container having a sterile access port, for example, an intravenous solution bag or vial having a stopper pierceable by a hypodermic injection needle. The formulations are preferably administered as repeated intravenous (i.v.), subcutaneous (s.c.), or intramuscular (i.m.) injections, or as aerosol formulations suitable for intranasal or intrapulmonary delivery (for intrapulmonary delivery see, e.g., EP 257,956).

The anti-angiogenic or the pro-angiogenic substances can also be administered in the form of sustained-released preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacr- ylate) as described by Langer et al., J. Biomed. Mater. Res., 15: 167-277 (1981) and Langer, Chem. Tech., 12: 98-105 (1982) orpoly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot.TM. (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37.degree. C., resulting in a loss of biological activity and possible changes in immunogenicity. Rational strategies can be devised for protein stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release anti-angiogenic or pro-angiogenic substances compositions also include liposomally entrapped polypeptides, like the netrin-1 polypeptides or the antibodies directed against the netrin-1 receptor or also the antibodies against netrin-1. Liposomes containing the polypeptide of interest are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamellar type in which the lipid content is greater than about 30 mol. % cholesterol, the selected proportion being adjusted for the optimal therapy.

The therapeutically effective dose of a anti-angiogenic or a pro-angiogenic substance will, of course, vary depending on such factors as the pathological condition to be treated (including prevention), the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect. The clinician will administer the anti-angiogenic or the pro-angiogenic substance of interest until a dosage is reached that achieves the desired effect for treatment of the condition in question.

With the above guidelines, the effective dose generally is within the range of from about 0.001 to about 1.0 mg/kg, more preferably about 0.01-1.0 mg/kg, most preferably about 0.01-0.1 mg/kg.

For non-oral use in treating human adult hypertension, it is advantageous to administer the anti-angiogenic or the pro-angiogenic substance in the form of an injection at about 0.01 to 50 mg, preferably about 0.05 to 20 mg, most preferably 1 to 20 mg, per kg body weight, 1 to 3 times daily by intravenous injection. For oral administration, a molecule based on the anti-angiogenic or the pro-angiogenic substance is preferably administered at about 5 mg to 1 g, preferably about 10 to 100 mg, per kg body weight, 1 to 3 times daily. It should be appreciated that endotoxin contamination should be kept minimally at a safe level, for example, less than 0.5 ng/mg protein. Moreover, for human administration, the formulations preferably meet sterility, pyrogenicity, general safety, and purity as required by FDA Office and Biologics standards.

The route of anti-angiogenic or pro-angiogenic substance administration is in accord with known methods, e.g., by injection or infusion by intravenous, intramuscular, intracerebral, intraperitoneal, intracerobrospinal, subcutaneous, intraocular, intraarticular, intrasynovial, intrathecal, oral, topical, or inhalation routes, or by sustained-release systems as noted below. The anti-angiogenic or the pro-angiogenic substances of interest also are suitably administered by intratumoral, peritumoral, intralesional, or perilesional routes, to exert local as well as systemic therapeutic effects. The intraperitoneal route is expected to be particularly useful, for example, in the treatment of ovarian tumors.

Examples of pharmacologically acceptable salts of molecules that form salts and are useful hereunder include alkali metal salts (e.g., sodium salt, potassium salt), alkaline earth metal salts (e.g., calcium salt, magnesium salt), ammonium salts, organic base salts (e.g., pyridine salt, triethylamine salt), inorganic acid salts (e.g., hydrochloride, sulfate, nitrate), and salts of organic acid (e.g., acetate, oxalate, p-toluenesulfonate).

The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT.TM. (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37.degree. C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Combination Therapies

The effectiveness of the anti-angiogenic or the pro-angiogenic substances in preventing or treating the disorder in question may be improved by administering the active agent serially or in combination with another agent that is effective for those purposes, either in the same composition or as separate compositions.

In addition, the anti-angiogenic or the pro-angiogenic substances used to treat cancer may be combined with cytotoxic, chemotherapeutic, or growth-inhibitory agents as identified above. Also, for cancer treatment, the anti-angiogenic or the pro-angiogenic substance of interest is suitably administered serially or in combination with radiological treatments, whether involving irradiation or administration of radioactive substances.

The effective amounts of the therapeutic agents administered in combination with the anti-angiogenic or the pro-angiogenic substances will be at the physician's or veterinarian's discretion. Dosage administration and adjustment is done to achieve maximal management of the conditions to be treated. For example, for treating hypertension, these amounts ideally take into account use of diuretics or digitalis, and conditions such as hyper- or hypotension, renal impairment, etc. The dose will additionally depend on such factors as the type of the therapeutic agent to be used and the specific patient being treated. Typically, the amount employed will be the same dose as that used, if the given therapeutic agent is administered without the anti-angiogenic or the pro-angiogenic substances.

Diseases or Conditions That May be Prevented or Treated with a Therapeutic Composition According to the Invention.

Diseases or conditions that may be prevented or treated with a therapeutic substance described in the present specification encompass all diseases or conditions wherein an imbalance or deregulation of the angiogenesis process is encountered, respectively the diseases or conditions wherein an inhibition of angiogenesis is desired and the diseases or conditions wherein an increase or promotion of angiogenesis is desired.

These diseases or conditions encompass those for which promotion of angiogenesis is desired, which include, without being limited to, treatment of a patient with a disease or a condition that is indicated by decreased vascularization, when a rapid wound healing is sought, peripheral vascular disease, hypertension, inflammatory vasculitides, Reynaud's disease and Reynaud's phenomenon, aneurysms, arterial restenosis, thrombophlebitis, lymphangitis, lymphedema, wound healing and tissue repair (especially hepatic and renal tissues), ischemia reperfusion injury, angina, myocardial infarctions such as acute myocardial infarctions, chronic heart conditions, heart failure such as congestive heart failure, and osteoporosis. Those diseases or conditions also include diabetes, arthritis, ischemia, anemia, a wound, gangrene or necrosis.

Most importantly, the conditions or diseases that are concerned are those for which angiogenesis is pathological and should be reduced or blocked.

These diseases or conditions include stroke, hemangioma, myocardial angiogenesis, plaque neovascularization, coronary collaterals, ischemic limb angiogenesis, atherosclerosis, leukocyte trafficking and recruitment, homeostasis, wound healing, vascular leaky syndrome, corneal diseases, rubeosis, neovascular glaucoma, macular degeneration, diabetic retinopathy, retinopathy of prematurity (ROP), retrolental fibroplasia, arthritis, including rheumatoid arthritis and osteoarthritis, diabetic neovascularization, macular degeneration, wound healing, peptic ulcer, fractures, keloids, vasculogenesis, hematopoiesis, ovulation, menstruation, placentation, polycyctic ovary syndrome, dysfunctional uterine bleeding, endometrial hyperplasia, endometriosis, failed implantation and subnormal fetal growth, myometrial fibroids and adenomyosis, ovarian hyperstimulation syndrome, solid tumors, carcinoma including adenocarcinoma, lymphoma, blastoma, melanoma, sarcoma, and leukemia, squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer,renal cell carcinoma, Wilms' tumors, basal cell carcinoma, melanoma, prostate cancer, vulval cancer, thyroid cancer, testicular cancer, esophageal cancer, head cancers, neck cancers, breast cancer, coloncancer, lung cancer, melanoma, ovarian cancer and cancers involving vascular tumors

These diseases or conditions include ocular neovascular disease, age-related macular degeneration, diabetic retinopathy, retinopathy of prematurity, corneal graft rejection, neovascular glaucoma, and retrolental fibroplasias. Other diseases associated with corneal neovascularization include, but are not limited to, epidemic keratocunjunctivitis, Vitamin A deficiency, contact lens overwear, atopic keratitis, superior limbic keratitis, pterygium kreatitis sicca, sjogrens, acne rosacea, phylectenulosis, syphilis, mycobacterial infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, rheumatois arthritis, systemic lupus, polyarteritis, Wegener's sarcoidiosis, scleritis, Stevens-Johnson disease, pemphigoid, radial keratotomy and corneal graft rejection.

These diseases or conditions also encompass those associated with retinal/choroidal neovascularization which include, but are not limited to, diabetic retinopathy, macular degeneration, sickle cell anemia, sracoid, syphilis, pseudoxanthoma elasticum, Paget's disease, vein occlusion, artery occlusion, carotid obstructive disease, chronic uveitis/vitritis, mycobaterial infections, Lyme's disease, systemic lupus erythematosis, retinopathy of prematurity, Eale's disease, Bechet's disease, infections causing retinitis or choroiditis, presumed ocular histoplasmosis, Best's disease, myopia, optic pits, Stargardt's disease, pars planitis, chronic etinal detachment, hyperviscosity syndromes, toxoplasmosis, trauma and post-laser complications. Other diseases include, but are not limited to, diseases associated with rubeosis, which causes neovascularisation of the angle, and diseases caused by abnormal proliferation of fibrovascular or fibrous tissue including all forms of proliferative vitreoretinopathy.

These diseases or conditions also include chronic inflammation, such as Crohn's disease and bacterial infections like Bartonellosis.

Most importantly, these diseases and conditions encompass cancers.

The family of benign and malignant vascular tumors are characterized by abnormal proliferation and growth of cellular elements s of the vascular system. For example, lymphangiomas are benign tumors of the lymphatic system that are congenital, often cystic, malformations of the lymphatics that usually occur in newborns. Cystic tumors tend to grow into the adjacent tissue. Cystic tumors usually occur in the cervical and axillary region. They can also occur in the soft tissue of the extremities. The main symptoms are dilated, sometimes reticular, structured lymphatics and lymphocysts surrounded by connective tissue. Lymphangiomas are assumed to be caused by improperly connected embryonic lymphatics or their deficiency. The result is impaired local lymph drainage. Griener et al., Lymphology, 4: 140-144 (1971).

As indicated earlier, the anti-angiogenic substances disclosed in the present dscription, would be useful in the prevention of tumor angiogenesis, a process which involves vascularization of a tumor to enable it to growth and/or metastasize. This process is dependent on the growth of new blood vessels. Examples of neoplasms and related conditions that involve tumor angiogenesis include breast carcinomas, lung carcinomas, gastric carcinomas, esophageal carcinomas, colorectal carcinomas, liver carcinomas, ovarian carcinomas, the comas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the the anti-angiogenic or the pro-angiogenic substances of the invention are expected to be useful in reducing the severity of AMD.

Healing of trauma such as wound healing and tissue repair is also a targeted use for the anti-angiogenic or the pro-angiogenic substances of the invention. Formation and regression of new blood vessels is essential for tissue healing and repair. This category includes bone, cartilage, tendon, ligament, and/or nerve tissue growth or regeneration, as well as wound healing and tissue repair and replacement, and in the treatment of burns, incisions, and ulcers.

The anti-angiogenic or the pro-angiogenic substances according to the invention may also be useful to promote better or faster closure of non-healing wounds, including without limitation pressure ulcers, ulcers associated with vascular insufficiency, surgical and traumatic wounds, and the like.

Ischemia-reperfusion injury is another indication. Endothelial cell dysfunction may be important in both the initiation of, and in regulation of the sequelae of events that occur following ischemia-reperfusion injury.

Rheumatoid arthritis is a further indication. Blood vessel growth and targeting of inflammatory cells through the vasculature is an important component in the pathogenesis of rheumatoid and sero-negative forms of arthritis.

Additional non-neoplastic conditions include psoriasis, diabetic and other proliferative retinopathies including retinopathy of prematurity, retrolental fibroplasia, neovascular glaucoma, comeal and other tissue transplantation and chronic inflammation. The present invention is further illustrated by, without in any way being limited to, the following examples.

### EXAMPLES

### A. MATERIAL AND METHODS

A.1. Recombinant Proteins, Immunohistochemistry

Recombinant proteins except bFGF (Bachem) were from R&D. For immunohistochemistry, lsolectinB4 (Sigma), streptavidin Cy-3 (Amersham), PECAM-1 (Pharmingen), anti-NRP-1 and anti-NRP-2 (R&D) and Cleaved caspase-3 (Cell Signaling) were used. Histology, immunohistochemistry, in situ hybridization and X-gal staining were performed as described in Yuan, L. et al. (Abnormal lymphatic vessel development in neuropilin 2 mutant mice. Development 129, 4797-806 (2002)). BrdU injections were performed as described in Yuan et al. (Supra); embryos were collected after three hours and stained (Yuan et al., Supra). Whole-mount isolectinB4 staining was described in Gerhardt, H. et al. (VEGF guides angiogenic sprouting utilizing endothelial tip cell filopodia. J Cell Biol 161, 1163-77 (2003)). Confocal images were acquired using a Leica TCS SP2 confocal miscroscope. Two independent observers performed counting of endothelial branch points manually. For each stage, three 1 mm×1 mm images taken from 4−/−, 4± and 3+/+embryos (4 litters) were counted. For quantification of BrdU-lacZ double-labeled endothelial cells, six sections from 4−/−, 4± and 2+/+embryos were counted using Metaview software (Princeton, version 5.0r6, 2002, US). Statistical analysis was done using Mann-Whitney test unless otherwise indicated. *: p<0.05.

A.2. RT-PCR

Genotyping of Unc5h2 mice was done using the following primers:

| | |
|---|---|
| Unc5h2: | |
| ACTAGAATGCTGTCCAGAC; | (SEQ ID N° 8) |
| AGAGGAGAGCAACGGATG, | (SEQ ID N° 9) |
| Plap: | |
| TGCACATGCTTTACGTGTG; | (SEQ ID N° 10) |
| CGCGTGTCGTGTTGCAC. | (SEQ ID N° 11) |

For expression studies in HUAEC or HUVEC we used the following primer sets:

| | |
|---|---|
| Unc5h1: | |
| AGCTGTCCCTTAATGCTGGT; | (SEQ ID N° 12) |
| AAGGCTGTGTACATAAGGCC, | (SEQ ID N° 13) |
| Unc5h2: | |
| ACTGGATCTTTCAGCTCAAG; | (SEQ ID N° 14) |
| AGTAATTCAGGTACCGGTCC | (SEQ ID N° 15) |
| Unc5h3: | |
| ATTTGCCGCTGCTGGATCCT; | (SEQ ID N° 16) |
| ACAACAAACCGTCCACAGCT, | (SEQ ID N° 17) |
| Unc5h4: | |
| GCCTCGAGTACTTGGTAAGT; | (SEQ ID N° 18) |
| TGTGTCATTCTCTGTAGGCC, | (SEQ ID N° 19) |
| Dcc: | |
| AACACTCTCAGTGGACCGAG; | (SEQ ID N° 20); |
| TCCTTAACTGAGTGGTCCTG, | (SEQ ID N° 21) |
| A2b: | |
| CTATGCTTACCGGAACCGAG | (SEQ ID N° 22) |
| ACCATGCCCGGCCGAATAAT, | (SEQ ID N° 23) |
| \overline{β}tubulin: | |
| GCTTCAAGGTTGGCATCAAC; | (SEQ ID N° 24) |
| TAGTATTCCTCTCCTTCTTC. | (SEQ ID N° 25) |

A.3. Endothelial Migration Assays

HUAEC and HUVEC (Promocell) between passage 3 and 7 were cultured in endothelial cell growth medium (ECGM, Promocell) containing 10% growth supplement (GS, Promocell). Transwell migration chambers (Costar) containing 8 μm-pore filters were coated with fibronectin (50 μg/ml); the upper wells were seeded with 5×10⁴ HUAEC or HUVEC. The lower chamber was seeded with confluent 293 or 293 Netrin-1 secreting cells (Shirasaki, R., Mirzayan, C., Tessier-Lavigne, M. & Murakami, F. Guidance of circumferentially growing axons by Netrin-dependent and -independent floor plate chemotropism in the vertebrate brain. Neuron 17, 1079-88 (1996)). Cells were left to migrate for 2 hours; cells remaining in the upper well were mechanically removed. Cells at the bottom side of the filter were fixed with 4% paraformaldehyde, washed and counted after Hoechst nuclear stain using Metaview software. For wound migration assays, confluent HUAEC starved overnight in 1% GS were wounded with a pipette tip. After 24 hours, cultures were photographed and two independent observers counted cells migrating into the wound area manually. For aortic ring assays, the abdominal aorta from 2month-old anaesthetized rats was excised, washed in ECGM and cut into small rings. Rings were placed in semi-solid collagen cultures (Spassky, N. et al. Directional guidance of oligodendroglial migration by class 3 semaphorins and netrin-1. J Neurosci 22, 5992-6004 (2002)) in ECGM containing 50 ng/ml of VEGF and sprouts were allowed to develop over a period of 5days. Recombinant Netrin-1 gradients (1 μg/μl) were applied using a micropipette. Individual sprouts were filmed under an inverted microscope (Leika) equipped with a digital camera (Princeton Coolsnap cf. Time-lapse images were acquired using Metaview software.

A.4. Intraocular and Hindbrain Injection

Intraocular injections were performed as described (Gerhardt, H. et al. VEGF guides angiogenic sprouting utilizing endothelial tip cell filopodia. J Cell Biol 161, 1163-77 (2003)), except that pups were anaesthetized using Ketamine-Xylazine and sacrificed three hours after the injection. All proteins were injected at 1 μg/μl except bFGF 200ng/μl. Pre-clustering of Netrin-1 with UNC5H2-Fc was done at a 1:1 ratio for 1 hour at 4° C. Number of injected eyes: Netrin-1: 5, Netrin-1/UNC5H2-Fc: 3, Netrin-4: 3, Flt-1-Fc: 2, bFGF: 2, BSA: 3, uninjected: 14. For hindbrain injections, E10.5 CD1 embryos were isolated in warm embryo culture medium (Sugiyama, D. et al. Erythropoiesis from acetyl LDL incorporating endothelial cells at the preliver stage. Blood 101, 4733-8 (2003)), injected using calibrated micropipettes and cultured for three hours in rat serum (Charles River) in a roller culture chamber (BTC Engineering, UK). Number of injected embryos: BSA: 4+/+, 3±, Netrin-1: 3+/+, 3±, 4−/− (three litters). Quantification was done on three 1 mm/1 mm images/retina or hindbrain.

### B. EXAMPLES

### Example 1

### The Netrin Receptor UNC5H2 is Selectively Expressed in the Vascular System

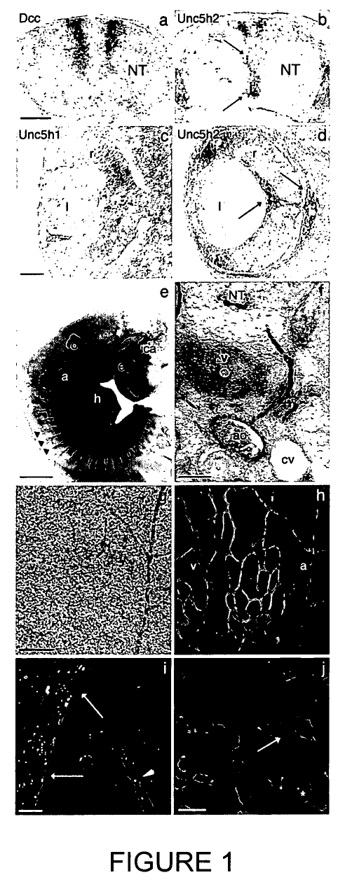

To assess the potential role of Netrin receptors in vascular development, we first examined their expression in the vascular system. In situ hybridizations with probes recognizing Unc5h1, Unc5h2 and Dcc were performed on sections from mouse embryos between E10.5 and E18.5. Dcc was not detected in endothelial cells, but was observed to be expressed in a variety of other locations that have been previously reported (Engelkamp, D. Cloning of three mouse Unc5 genes and their expression patterns at mid-gestation. Mech Dev 118, 191-7 (2002); Barallobre, M. J. et al. Aberrant development of hippocampal circuits and altered neural activity in netrin 1-deficient mice. Development 127, 4797-810 (2000); Deiner, M. S. et al. Netrin-1 and DCC mediate axon guidance locally at the optic disc: loss of function leads to optic nerve hypoplasia. Neuron 19, 575-89 (1997); Jiang, Y., Liu, M. T. & Gershon, M. D. Netrins and DCC in the guidance of migrating neural crest-derived cells in the developing bowel and pancreas. Dev Biol 258, 364-84 (2003)), including in commissural neurons in the dorsal neural tube (Keino-Masu, K. et al. Deleted in Colorectal Cancer (DCC) encodes a Netrin receptor. Cell 87, 175-85 (1996)) (FIG. 1a). Expression of Unc5h1 was observed in various neural and non-neural structures (Leonardo, E. D. et al. Vertebrate homologues of C. elegans UNC-5 are candidate netrin receptors. Nature 386, 833-8 (1997); Engelkamp, D. Cloning of three mouse Unc5 genes and their expression patterns at mid-gestation. Mech Dev 118, 191-7 (2002); Barrett, C. & Guthrie, S. Expression patterns of the netrin receptor UNC5H1 among developing motor neurons in the embryonic rat hindbrain. Mech Dev 106, 163-6 (2001)), and data not shown) including the otic placode and olfactory system, but not in the vasculature. Importantly, Unc5h2, while showing low expression in the ventricular zone of the neural tube, strongly labeled capillaries in the perineural vascular plexus and invading the neural parenchyme (FIG. 1b). On sections through the developing eye, Unc5h2 was expressed in the dorsal aspect of the developing neural retina, but also strongly labeled the developing intra-ocular vasculature (FIG. 1d). Both Dcc (not shown) and Unc5h1 were expressed in the neural retina, but could not be detected on developing capillaries (FIG. 1c). In summary, Unc5h2 was expressed in developing blood vessels in all tissues and at all stages examined, while neither of the other two Netrin receptors were detected in the vascular system. Moreover, the expression of Unc5h2 appeared largely restricted to the vascular system, although expression in some other neural and non-neural structures was also observed, as previously reported (Leonardo, E. D. et al. Vertebrate homologues of C. elegans UNC-5 are candidate netrin receptors. Nature 386, 833-8 (1997); Engelkamp, D. Cloning of three mouse Unc5 genes and their expression patterns at mid-gestation. Mech Dev 118, 191-7 (2002)).

We extended this expression analysis, taking advantage of an Unc5h2 knock-out mouse we generated to analyze the function of Unc5h2 (see below). The knock-out method we used resulted in the introduction of a beta-galactosidase reporter into the Unc5h2 locus, such that X-gal staining results in labeling of cells normally expressing unc5h2 in animals that are homozygous or heterozygous for the mutation. In what follows, we will interpret beta-galactosidase expression to reflect Unc5h2 expression, which is justified by the complete concordance of expression with that observed by in situ hybridization using an Unc5h2 probe. Whole-mount X-gal staining of E10.5 heterozygous embryos confirmed that Unc5h2 was mainly expressed in the vascular system, with neural expression restricted until E12.5 to the developing retina and otic placode (FIG. 1e). Interestingly, expression was not uniform throughout the vasculature: expression was observed in the dorsal aorta, intersomitic arteries and internal carotid artery, whereas superficially localized veins were negative. Sectioning through the trunk region confirmed that Unc5h2 is specifically expressed by arteries, not veins (FIG. 1f). To determine precisely the cellular expression of Unc5h2 in the developing vascular system, we examined the retinal vasculature of postnatal Un5h2 heterozygous pups (FIG. 1g-j). Double staining with X-gal and isolectinB4 confirmed that arteries, but not veins expressed Unc5h2 (FIG. 1g, h). Moreover, expression in capillaries extended approximately halfway between the arteries and veins. High magnification double staining of retinal vessels with isolectinB4 and an anti-beta-galactosidase antibody showed that endothelial cells, but not pericytes expressed Unc5h2 (FIG. 1i). Interestingly, endothelial tip cells localized at the ends of developing capillary sprouts strongly expressed Unc5h2 (FIG. 1i, j). Tip cells were labeled on both the arterial and the venous side of capillaries. In summary, Unc5h2 was expressed by arterial and a subset of capillary endothelial cells, as well as by both arterial and venous tip cells.

### Example 2

### Generation of Unc5h2 Knockout Mice

To examine the function of Unc5h2 in developing endothelial cells, we studied the effect of loss of function of Unc5h2 using the knock-out mouse we had generated. The mutant allele was generated in embryonic stem (ES) cells using homologous recombination to direct the so-called “secretory-trap” vector (Leighton, P. A. et al. Defining brain wiring patterns and mechanisms through gene trapping in mice. Nature 410, 174-9 (2001)) into the Unc5h2 locus at an intron between the second and third immunoglobulin (Ig) domains of the UNC5H2 extracellular domain (Supplementary FIG. 1). The secretory trap vector possesses a splice acceptor followed by sequences coding for, in succession, a linker protein, a transmembrane domain, beta-geo (a beta-galactosidase/neomycin phosphotransferase fusion), internal ribosome entry site and placental alkaline phosphatase. The presence of this vector causes upstream exons to splice to the vector sequences, resulting in the generation of a fusion protein between the first two Ig domains of UNC5H2 and the vector-coded sequences. This fusion protein is retained in an intracellular compartment of the cells (Leighton, P. A. et al. Defining brain wiring patterns and mechanisms through gene trapping in mice. Nature 410, 174-9 (2001)), so that the mutation is predicted to be a null or near null allele. The mutation was backcrossed to both CD1 and C57BU6J backgrounds. All heterozygous animals were indistinguishable from wild type, healthy and bred normally. On a BL/6 background, homozygous embryos were recovered at Mendelian frequencies until E10. At this point, they appeared growth retarded and often presented failure of cephalic neural tube closure (not shown). On an outbred CD1 background, however, growth retardation and failure of neural tube closure were not observed (not shown); homozygotes were recovered at Mendelian frequencies until E12.5. Their death was due to heart failure, as indicated by accumulation of blood in the venous circulation. No obvious cardiac malformations were detected by histological examination (data not shown); a possible cause of heart failure was increased peripheral resistance due to the abnormal arterial vasculature (described below), although we have not excluded alternative possibilities, such as failure in the development of the cardiac conduction system. The vascular phenotype of embryos described below was very similar on both genetic backgrounds (data not shown), although only limited characterization was performed on the BU6 background.

### Example 3

### Increased Vessel Branching and Tip Cell Filopodia Extension in Unc5h2 Mutant Embryos

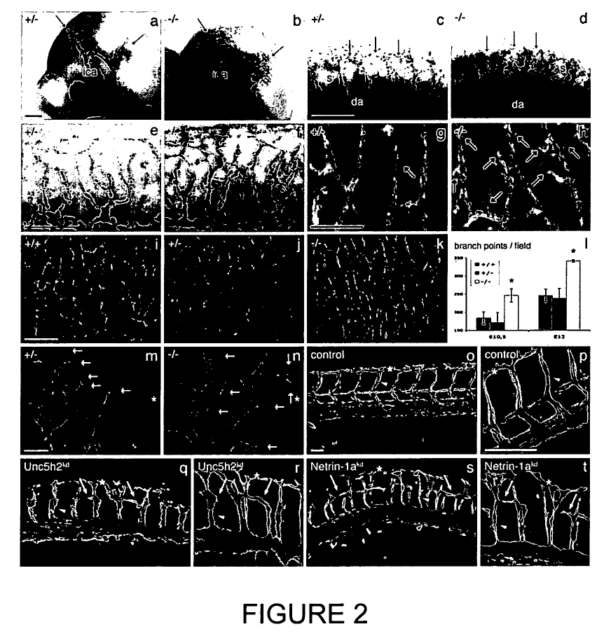

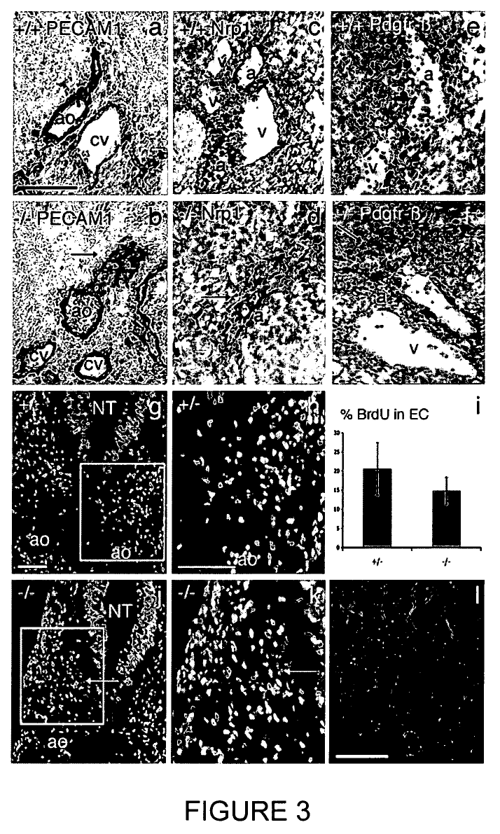

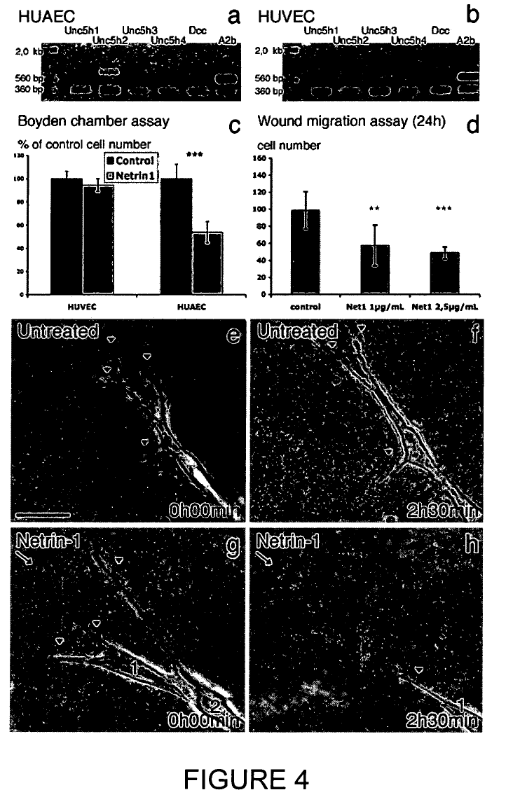

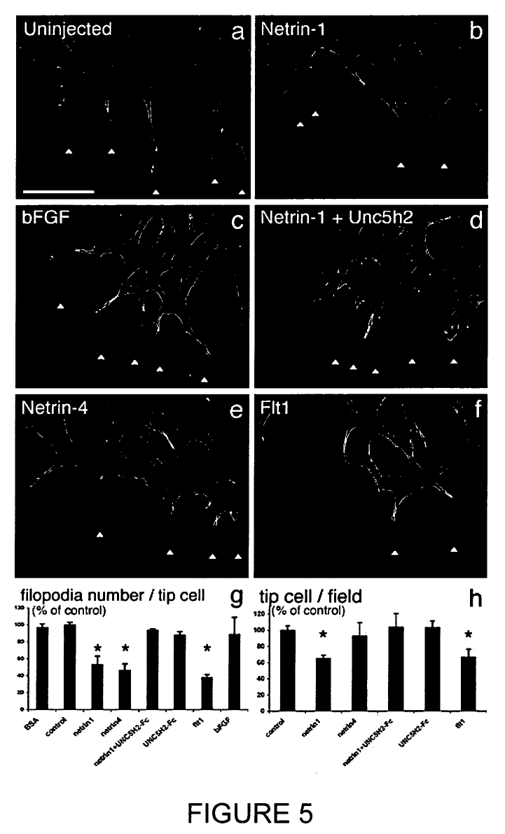

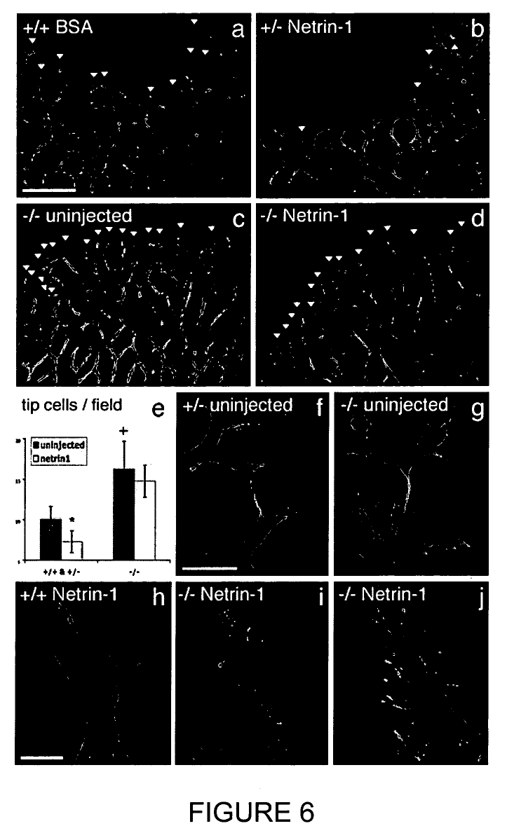

Homozygous Unc5h2 mutant embryos formed a normal primary vascular plexus, which also remodeled normally into arteries and veins (FIGS. 2, 3). Examination of whole-mount X-gal stained embryos revealed an increased number of branches of the internal carotid artery in mutant embryos (FIG. 2a, b). Ectopic and supernumerary capillary sprouts were Is also observed in the somitic region of mutant embryos (FIG. 2c, d). Even more obvious was increased vessel branching within the embryonic nervous system (FIG. 2e, f). To confirm that capillary branching in Unc5h2 mutants was increased, we performed whole-mount staining of hindbrains with isolectinB4. Double labeling of lacZ and isolectinB4 was observed in the vast majority of brain capillaries (FIG. 2g, h). Capillaries of mutant brains appeared thinner and more highly branched compared to vessels in stage-matched wild-type or heterozygous embryos (FIG. 2i-k). Counting of the number of branch points of isolectinB4-stained whole-mount preparations revealed an increase of about 40% in the mutants as compared to heterozygous or wild-type hindbrain capillaries (FIG. 21).

Similar results were obtained when counting neural tube vessels (not shown). Interestingly, filopodial extension from Unc5h2 expressing tip cells appeared more abundant in Unc5h2 mutant capillaries as compared to heterozygotes (FIG. 2g, h). High-magnification confocal microscopy confirmed that filopodial extension from tip cells was strikingly increased in Unc5h2 mutant embryos (FIG. 2m, n).

To determine if vessel branching was selectively deficient in Unc5h2 mutants, embryos were sectioned and stained with different markers. Staining with the pan-vascular marker PECAM-1 showed that the lumen of abnormally branched mutant arteries was often collapsed or irregularly shaped (FIG. 3a, b). Affected arteries never included the dorsal aorta, but did include its side branches (FIG. 3). In spite of their abnormal morphology, homozygous mutant vessels expressed arterial markers including ephrinB2 (not shown) and neuropilin-1 (FIG. 3c, d) normally. No change in expression of venous markers neuropilin-2 and EphB4 was observed in the mutant embryos (not shown). The vessel wall of mutant arteries also formed normally, as indicated by expression of Pdgfr-62 (FIG. 3e,f) or anti-smooth muscle actin staining (not shown). Proliferation of mutant endothelial cells, as judged by computer-assisted counting of BrdU-lacZ-double-positive cells, was not significantly different between heterozygous and homozygous embryos (FIG. 3g-k). Apoptosis of endothelial cells was virtually undetectable in embryos of either genotype (FIG. 31 and data not shown). Taken together, these observations suggest that the primary defect in Unc5h2 mutant vessels is a branching defect.

### Example 4

### Reduced Endothelial Migration and Tip Cell Filipodia Retraction in Response to Netrin-1

Since Unc5h2-deficient vessels exhibit larger numbers of extending filopodia, we predict that the normal function of UNC5H2 should be to negatively regulate filopodia extension in the vascular system. Filopodial retraction might ultimately lead to a reduction in cell migration; we thus examined the activity of the UNC5H2 ligand Netrin-1 on endothelial cell migration in vitro. To identify cell lines that might be responsive to Netrin-1, we examined expression of mRNAs for Netrin receptors in primary human umbilical artery (HUAEC) and vein (HUVEC) endothelial cells by RT-PCR (FIG. 4a, b). Both HUAEC and HUVEC expressed mRNA for the adenosine2b receptor (Corset, V. et al. Netrin-1-mediated axon 5 outgrowth and cAMP production requires interaction with adenosine A2b receptor. Nature 407, 747-50 (2000)) at similar levels. Dcc, and Unc5h1, 3 and 4 were not detected in HUAEC or HUVEC, whereas Unc5h2 was strongly expressed in HUAEC, and at low levels in HUVEC. Thus, the expression of Netrin receptors on these cells in vitro was similar to that in lo vivo. We first subjected these cells to transwell migration tests in the presence of medium conditioned by stably transfected 293 cells secreting Netrin-1 (Shirasaki, R., Mirzayan, C., Tessier-Lavigne, M. & Murakami, F. Guidance of circumferentially growing axons by Netrin-dependent and—independent floor plate chemotropism in the vertebrate brain. Neuron 17, 1079-88 (1996)) or control 293 cells (FIG. 4c). Western blotting indicated that the Netrin-producing cells secreted about 2.5 μg/ml Netrin-1 (not shown), sufficient to maximally stimulate UNC5H receptors (Leonardo, E. D. et al. Vertebrate homologues of C. elegans UNC-5 are candidate netrin receptors. Nature 386, 833-8 (1997)). HUVEC showed no change in their migratory response in the presence of Netrin-1 (FIG. 4c). In contrast, migration of HUAEC in the presence of Netrin-1 was reduced by about 40% (FIG. 4c). We next tested “wounded” confluent HUAEC cultures. The cells were left for 24hours to migrate into the wounded area, in the presence of recombinant Netrin-1 (FIG. 4d). Cell migration was decreased in a dose-dependent manner in the presence of Netrin-1 (FIG. 4d). Thus, Netrin-1 showed inhibitory effects on the migration of endothelial cells expressing UNC5H2 in vitro, consistent with a possible negative role in filopodial extension.

To directly test for effects of Netrin-1 on filopodial extension, we used aortic ring sprouting assays (FIG. 4e-h). Time-lapse video-microscopy of unmanipulated sprouting endothelial tip cells over a period of two hours showed little or no net forward or reverse movement of the tip over this short period (FIG. 4e, f). In contrast, exposure of endothelial tip cells to a gradient of Netrin-1 resulted in clear retraction of the tip cell filopodia and backward movement of the tip cell (FIG. 4g, h).

To examine whether Netrin-1 could affect filopodial extension in vivo, we performed intra-ocular injections of recombinant protein into postnatal day5 (P5) mice, followed by analysis of the retinal vasculature. Compared to uninjected control eyes, Netrin-1 injected eyes showed a dramatic decrease in filopodial extension over the entire angiogenic front (FIG. 5a, b). Both the length and number of filopodia as well as the number of filopodia-extending cells decreased, such that the overall appearance of the Netrin-1 injected angiogenic front appeared smooth compared to the spiky control front (FIG. 5a, b). Quantification of the number of filopodia showed a statistically significant difference between Netrin-1 and control eyes (p<0.05, student's t-test). Filopodia retraction induced by Netrin-1 was specific, as shown by lack of significant effect on filopodia following injection of control protein (BSA) (not shown) or of the growth factor bFGF, a protein which, like Netrin-1, is highly basic and binds heparin tightly (FIG. 5c). Filopodial retraction induced by Netrin-1 could be completely neutralized by pre-incubation of Netrin-1 protein with recombinant UNC5H2-Fc (FIG. 5d), a fusion of the constant region of human IgG with the ectodomain of UNC5H2 which is known to bind Netrin-1 (Leonardo, E. D. et al. Vertebrate homologues of C. elegans UNC-5 are candidate netrin receptors. Nature 386, 833-8 (1997)). To test whether other members of the Netrin family had the capacity to mediate filopodial retraction, we injected recombinant Netrin-4 (Koch, M. et al. A novel member of the Netrin family, beta-Netrin, shares homology with the beta chain of laminin: identification, expression, and functional characterization. J Cell Biol 151, 221-34 (2000).), which also induced statistically significant (p<0.05) filopodial retraction (FIG. 5e). Acute sequestration of the positive regulator VEGF by injection of soluble Flt-1 (Gerhardt, H. et al. VEGF guides angiogenic sprouting utilizing endothelial tip cell filopodia. J Cell Biol 161, 1163-77 (2003)) (FIG. 5f) was as effective at stimulating filopodia retraction as was injection of Netrins (p<0.001).

### Example 5

### Netrin-1-Induced Tip Cell Filopodial Retraction is Lost in Unc5h2 Mutants

To assess whether filopodial retraction induced by Netrin-1 is mediated by signaling through UNC5H2, we performed injections of recombinant proteins into hindbrains of E10.5 Unc5h2 mutant embryos, followed by 3-hour embryo culture and analysis of the vasculature. Analysis of tip cell morphology was performed at the dorsal side of the hindbrain, furthest removed from the floorplate, the site of endogenous Netrin-1 production (Kennedy, T. E., Serafini, T., de la Torre, J. R. & Tessier-Lavigne, M. Netrins are diffusible chemotropic factors for commissural axons in the embryonic spinal cord. Cell 78, 425-35 (1994)). Injection of Netrin-lin wild-type or heterozygous embryos resulted in a striking reduction of filopodial extension from tip cells compared to uninjected or BSA-injected controls (FIG. 6a, b). As already observed in the eye, the dorsal angiogenic front of Netrin-1 injected capillaries appeared smooth, in contrast to its spiky appearance in uninjected or BSA-injected embryos. At high magnification, residual tip cells in wild-type or heterozygous Netrin-1 injected embryos showed few remaining filopodia (FIG. 6h); the number of tip cells extending filopodia in those animals was decreased by 30% compared to uninjected controls (FIG. 6e).

The dorsal angiogenic front in uninjected Unc5h2 mutant hindbrains showed more tip cells than wild-type or heterozygous controls, reflecting the increased capillary branching in these embryos (FIG. 6c, e). At high magnification, increased filopodial extension from dorsal tip cells could be observed in uninjected mutant embryos compared to controls (FIG. 6f, g). Strikingly, Netrin-1 injection into Unc5h2 mutant embryos did not produce any obvious effects on capillary morphology (FIG. 6c-e). The number of filopodia-extending tip cells was not significantly altered by Netrin-1 injection (FIG. 6d, e). Filopodial extension in Unc5h2 mutant tip cells was unaffected by Netrin-1 (FIG. 6i, j). Taken together, these observations suggest that Netrin-1-induced filopodial retraction of endothelial tip cells is mediated by UNC5H2 signaling.

1. A method for regulating angiogenesis in a subject in need thereof, comprising administering to said subject an effective amount of a substance capable of modulating the activity of a netrin-1 receptor.

2. The method of claim 1, wherein said substance is capable of promoting the netrin-1 receptor activity, and wherein the administration of said substance inhibits angiogenesis in said subject.

3. The method of claim 2, wherein said substance selectively binds to the netrin-1 receptor.

4. The method of claim 3, wherein said substance comprises netrin-1 or a netrin-1 fragment.

5. The method of claim 3, wherein said substance comprises a polypeptide capable of specifically binding to the extracellular domains of the netrin-1 receptor.

6. The method of claim 3, wherein said substance comprises an anti-netrin-1 receptor agonist antibody.

7. The method of claim 1, wherein said netrin-1 receptor is UNC5B.

8. The method of claim 1, wherein said substance is capable of inhibiting the netrin-1 receptor activity and wherein the administration of said substance promotes angiogenesis in said subject.

9. The method of claim 8, wherein said substance is capable of interfering the binding of netrin-1 to the netrin-1 receptor.

10. The method of claim 8, wherein said substance comprises a soluble form of the netrin-1 receptor, or a peptide fragment thereof.

11. The method of claim 10, wherein said substance comprises a fusion protein between the soluble netrin-1 receptor and the Fc portion of an immunoglobulin.

12. The method of claim 8, wherein said substance comprises an anti-netrin-1 neutralizing antibody.

13. The method of claim 8, wherein said substance comprises an anti-netrin-1 receptor antibody that blocks the binding of netrin-1 to the netrin-1 receptor.

14. The method of claim 8, wherein said substance decreases the level of netrin-1 receptor expression.

15. The method of claim 14, wherein said substance comprises a netrin-1 receptor-specific antisense polynucleotide.

16. The method of claim 8, wherein said substance decreases the level of netrin-1 expression.

17. The method of claim 8, wherein said substance comprises a netrin-1 -specific antisense polynucleotide.

18. A method for the screening of a candidate substance for its anti-angiogenic activity, wherein said method comprises the steps of:
c) providing a candidate substance; and
d) assaying said candidate substance for its ability to bind to a netrin-1 receptor.

19. The method of claim 18, further comprising the steps of:
c) selecting positively said substance if it binds to the netrin-1 receptor; and
d) assaying the candidate substance positively selected at step c) for its ability to promote angiogenesis in vitro or in vivo.

20. The method of claim 19, wherein at step d) said candidate substance is assayed for its ability to inhibit filopodial extension of endothelial cells in vitro or in vivo.

21. A method for the screening of a substance that promotes angiogenesis, comprising the steps of:
a) providing a candidate substance; and
b) assaying said candidate substance for its ability to negatively modulates the netrin-1 receptor activity.

22. The method of claim 21, wherein at step b) said candidate substance is assayed for its ability to block the binding between netrin-1 and the netrin-1 receptor.

23. The method of claim 21, wherein at step b) said candidate substance is assayed for its ability to decrease the netrin-1 receptor expression.

24. A pharmaceutical composition for preventing or treating a condition or a disease associated with undesirable neovascularization comprising an anti-angiogenic substance that has been selected according to claim 18.

25. The pharmaceutical composition according to claim 24, further comprising an effective amount of a second substance with anti-angiogenic activity.

26. A pharmaceutical composition for preventing or treating a condition or a disease associated with an insufficient vascular supply comprising a pro-angiogenic substance that has been selected according to claim 21.
